(19) United States
(12) Patent Application Publication
(10) Pub. No.: US 2006/0240044 A1
(43) Pub. Date: Oct. 26, 2006

(54) Streptococcal superantigens spe-l and spe-m

(76) Inventors: John D. Fraser, Meadowbank (NZ); Thomas K. Proft, Mission Bay (NZ)

Correspondence Address: EDWARDS & ANGELL, LLP, P.O. BOX 55874, BOSTON, MA, 02205, US

(21) Appl. No.: 10/513,713

(22) PCT Filed: May 9, 2003
(86) PCT No.: PCT/NZ03/00086

§ 371 Date: Aug. 11, 2005

(30) Foreign Application Priority Data
May 9, 2002 (NZ) 518882
May 17, 2002 (NZ) 519052

(51) Int. Cl. A61K 39/09; C07H 21/04; C12N 15/74; C12N 1/21; C07K 14/315

(52) U.S. Cl. 424/244.1; 435/69.3; 435/252.3; 435/471; 530/350; 536/23.7

(57) ABSTRACT

The invention provides superantigens SPE-L and SPE-M, and their functional equivalents. In addition, the invention relates to nucleic acid molecules encoding the superantigens and/or their functional equivalents, and variant nucleic acids. The invention also provides diagnostic and therapeutic applications based on such superantigens and nucleic acid molecules.

### TECHNICAL FIELD

This invention relates to superantigens, and to their use, including in diagnosis, serotyping and/or treatment of disease.

### BACKGROUND ART

Bacterial superantigens are the most potent T cell mitogens known and are believed to be involved in pathogenicity and virulence. They stimulate large numbers of T cells by directly binding to MHC class II and T cell Receptor (TcR) molecules via the TcRVβ domain. Because they override the normally exquisite MHC restriction phenomenon of T cell antigen recognition, they are prime candidates for either causing the onset of autoimmune diseases or exacerbating an existing autoimmune disorder. Specifically, superantigens cause the production of high systemic levels of proinflammatory cytokines (TNF-α, IL-Iβ) and T cell mediators (IL-2, INF γ) that can cause hypertension, fever and shock.

The applicants have identified genes coding for two novel superantigens from S. pyogenes. It is broadly to these superantigens and polynucleotides encoding them that the present invention is directed.

### SUMMARY OF THE INVENTION

The invention generally relates to a superantigen selected from either SPE-L or SPE-M, or a functionally equivalent variant thereof, as well as to polynucleotide molecules including a sequence encoding a superantigen chosen from SPE-L or SPE-M, or a functionally equivalent variant thereof. Further the invention provides methods of subtyping Streptococci and diagnosis on the basis of superantigen genotype including detection of the presence of any or all of the above two superantigens or the corresponding polynucleotides, or a functionally equivalent variant thereof. In further aspects, the invention provides constructs including any of the above superantigens (or superantigen variants) bound to a cell-targeting molecule, which is preferably a tumour-specific antibody, and pharmaceutical compositions for therapy or prophylaxis including a superantigen or superantigen variant as described above linked to cell targeting molecule.

More specifically, in one aspect, the present invention provides a superantigen selected from either SPE-L or SPE-M, or a functionally equivalent variant thereof.

In another aspect, the invention provides a nucleic acid encoding a superantigen selected from either SPE-L or SPE-M, or a functionally equivalent variant thereof.

In a related aspect, the invention provides a nucleic acid comprising the sequence SEQ ID NO:1, or a variant thereof.

In a further related aspect, the invention provides a nucleic acid comprising the sequence SEQ ID NO:3, or a variant thereof.

In other aspects, the invention provides nucleic acids selected from the group consisting of:
SEQ ID NO: 5
SEQ ID NO: 6
SEQ ID NO: 7
SEQ ID NO: 8
SEQ ID NO: 9
SEQ ID NO: 10
SEQ ID NO: 11
SEQ ID NO: 12

In yet a further aspect, the invention provides nucleic acid constructs comprising one or more of the nucleic acids herein before described.

In another aspect, the invention provides a method of determining in a sample the presence or absence of the superantigens SPE-L and/or SPE-M, or functional equivalents thereof, comprising at least the steps of:
Providing a sample to be tested; and
Determining whether or not either or both of the superantigens, or their functional equivalents are present.

In a related aspect, the invention provides a method of determining in a sample the presence or absence of nucleic acid molecules encoding of the superantigens.SPE-L and/or SPE-M, or functional equivalents thereof, comprising at least the steps of:
Providing a sample to be tested; and
Determining whether or not nucleic acid molecules encoding either or both of the superantigens, or their functional equivalents are present.

In a further aspect, the invention provides a method of subtyping Streptococcus in a sample, the method comprising at least the steps of:
Providing a sample to be tested; and
Determining whether or not either or both of SPE-L or SPE-M, or their functional equivalents are present.

In a related aspect, the invention provides a method of subtyping Streptococcus in a sample, the method comprising at least the steps of:
Providing a sample to be tested; and
Determining whether or not nucleic acid molecules encoding either or both of the superantigens, or their functional equivalents are present.

In yet a further aspect, the invention provides a method of diagnosing infection of a subject with S.pyogenes, the method comprising at least the steps of:
Providing a sample from a subject to be tested; and,
Determining whether or not either or both of SPE-L or SPE-M, or their functional equivalents are present.

In a related aspect, the invention provides a method of diagnosing infection of a subject with S.pyogenes, the method comprising at least the steps of:
Providing a sample from a subject to be tested; and,
Determining whether or not nucleic acid molecules encoding either or both of the superantigens, or their functional equivalents are present.

Preferably, the purpose of diagnosing infection of a subject with S.pyogenes M-types M28, M41, M56, M59, M89, M80 and/or M92.

In a further aspect, the invention provides a method of determining whether or not a subject has been exposed to SPE-L and/or SPE-M comprising at least the steps of:
Providing a sample from a subject to be tested;
Determining whether or not the sample contains antibodies specific to SPE-L and/or SPE-M.

In another aspect, the invention provides a construct which comprises a superantigen or variant thereof as claimed in claim 1 and a cell-targeting molecule.

In another aspect, the invention provides a pharmaceutical composition which comprises the above construct.

In further aspects, the invention provides: an antibody which binds SPE-L or a functionally equivalent variant thereof; and, an antibody which binds SPE-M or a functionally equivalent variant thereof.

In additional aspects, the invention provides nucleic acid molecules which hybridises to polynucleotides SEQ ID NO:1 or SEQ ID NO:3.

In yet further aspects, the invention provides a kit which includes a nucleic acid molecule of the invention.

Other aspects of the invention will be apparent from the description provided below, and from the appended claims.

### DESCRIPTION OF DRAWINGS

While the invention is broadly defined above, it further includes embodiments of which the following description provides examples. It will also be better understood with reference to the following drawings:

FIG. 1: Multiple alignment of superantigen protein sequences. The protein sequence of mature streptococcal superantigens were aligned using the ClustaIX computer program. The clear box near the C-terminus represents a primary zinc binding motif, a common feature of all toxins shown. SePE-L and SePE-M are from Streptococcus equi (S. equi) and are homologues (or possibly orthologues) to SPE-L and SPE-M, respectively.

FIG. 2: Genotyping of streptococcal isolates

A. 40 S. pyogenes isolates (38 from New Zealand, 1 from U.K., 1 from France and 1 ATCC reference strain) were genotyped. The results are based on PCR analysis using purified genomic DNA and specific primers for each sag gene. The primers for spe-1 and spe-m were designed using the DNA sequences of the orthologous sepe-1 and sepe-m genes from S. equi. Site and disease abbreviation are as follows: ts, throat site; ws, wound site; sk, skin; ps, pus site; hvs, high vaginal site; bc, blood culture; ST, sore throat; RF, rheumatic fever; AGN, acute glumerulonephritis; T carriage, throat carriage; STSS, streptococcal toxic shock; n.d., not determined.

B. Genotyping of 11 S. pyogenes M89 isolates collected in New Zealand

FIG. 3: The nucleotide sequence of the portion of the spe-1 gene (SEQ ID NO. 1) coding the mature SPE-L superantigen (SEQ ID NO. 2). The gene was cloned from S. pyogenes M89 (isolate 10846) using DNA primers designed from the DNA sequence of the orthologous sepe-1 gene from S. equi. The DNA sequence of spe-1 was analysed using a Licor automated DNA sequencer (model 4200).

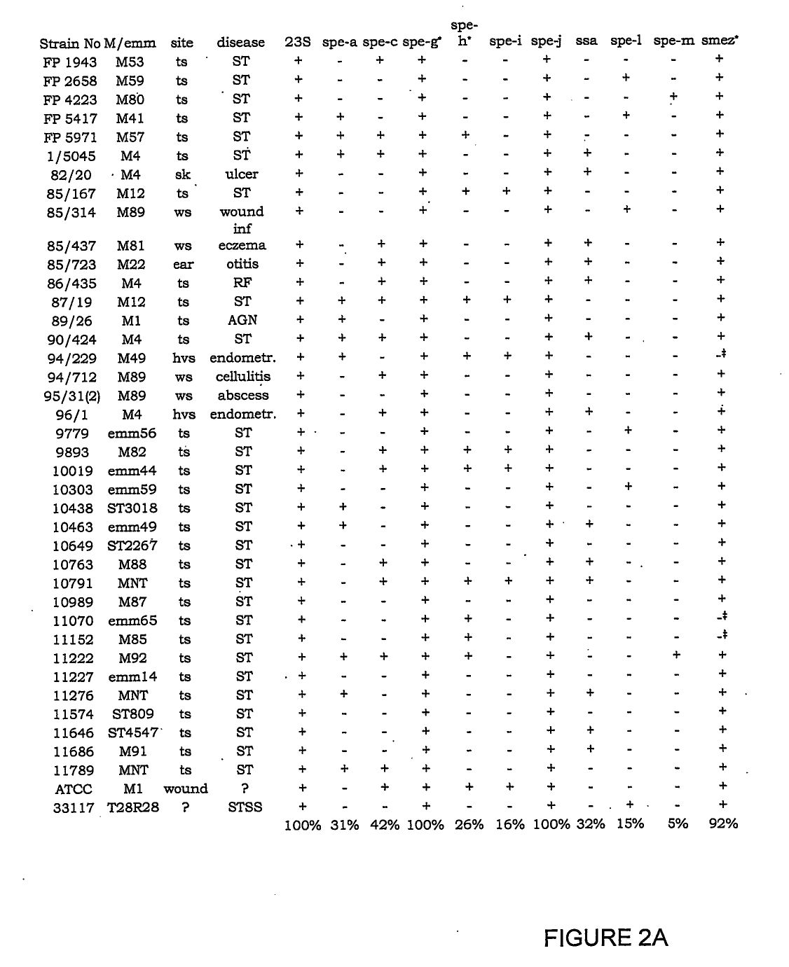

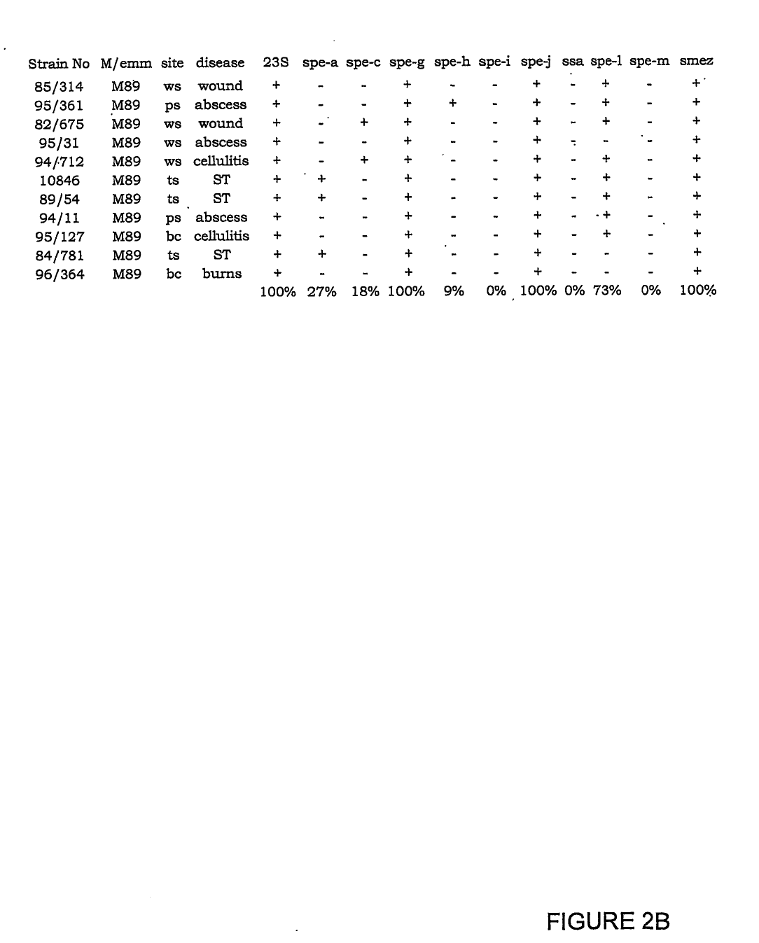

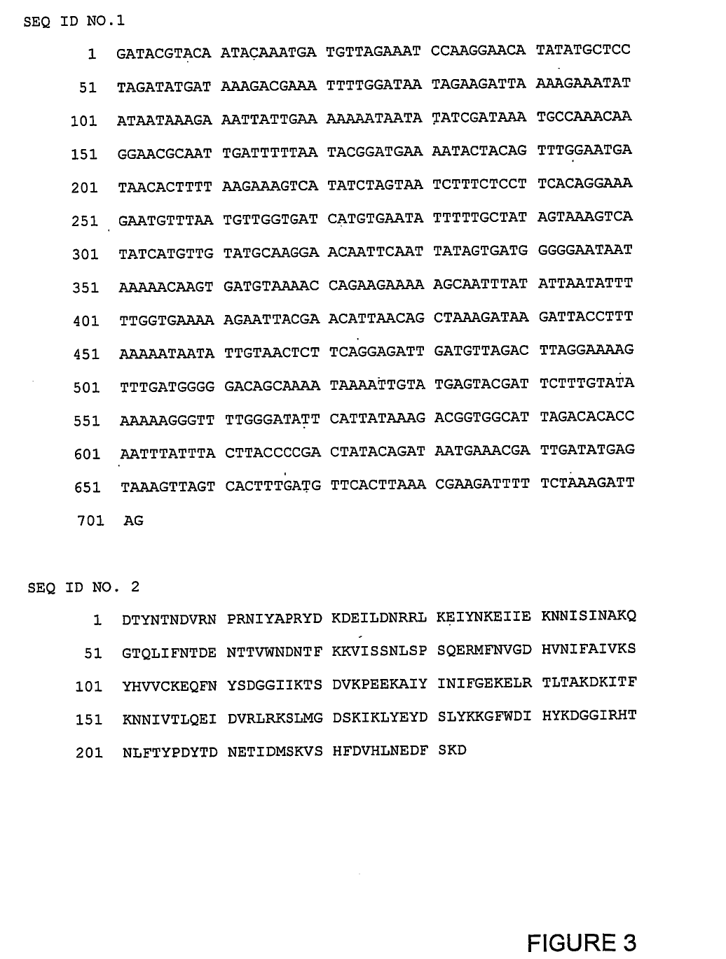

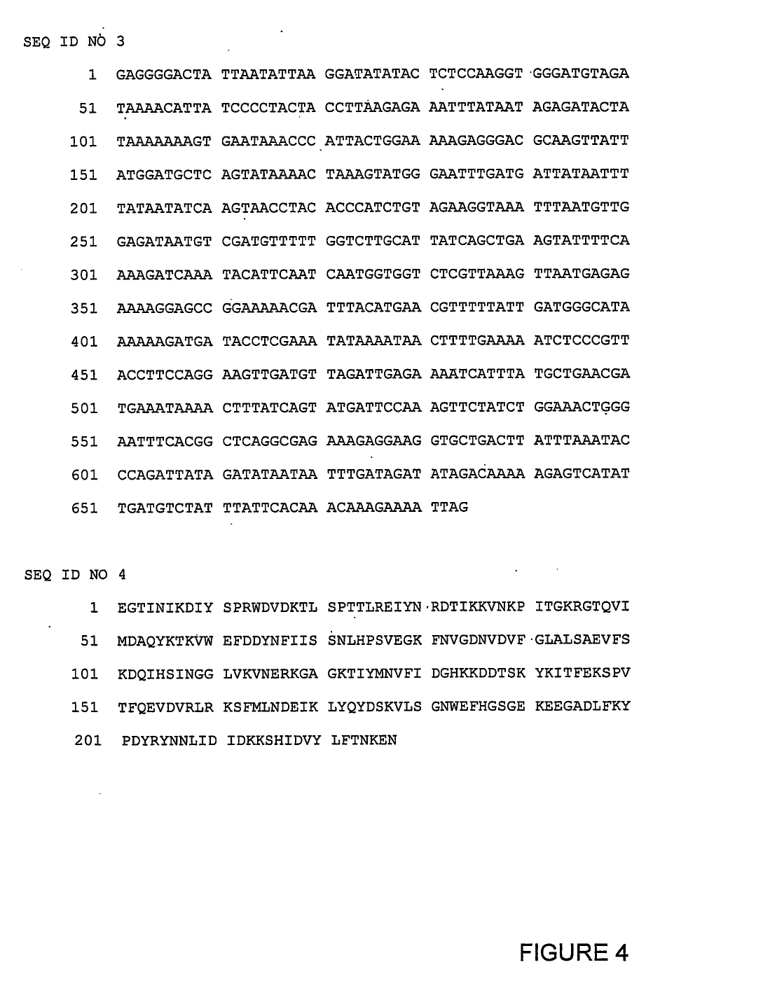

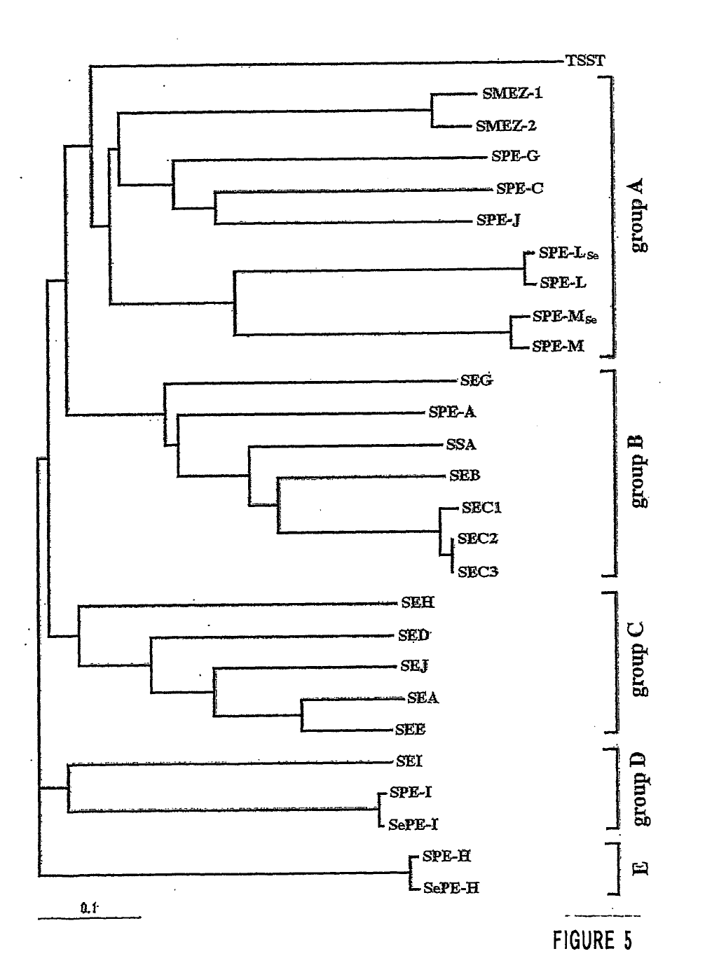

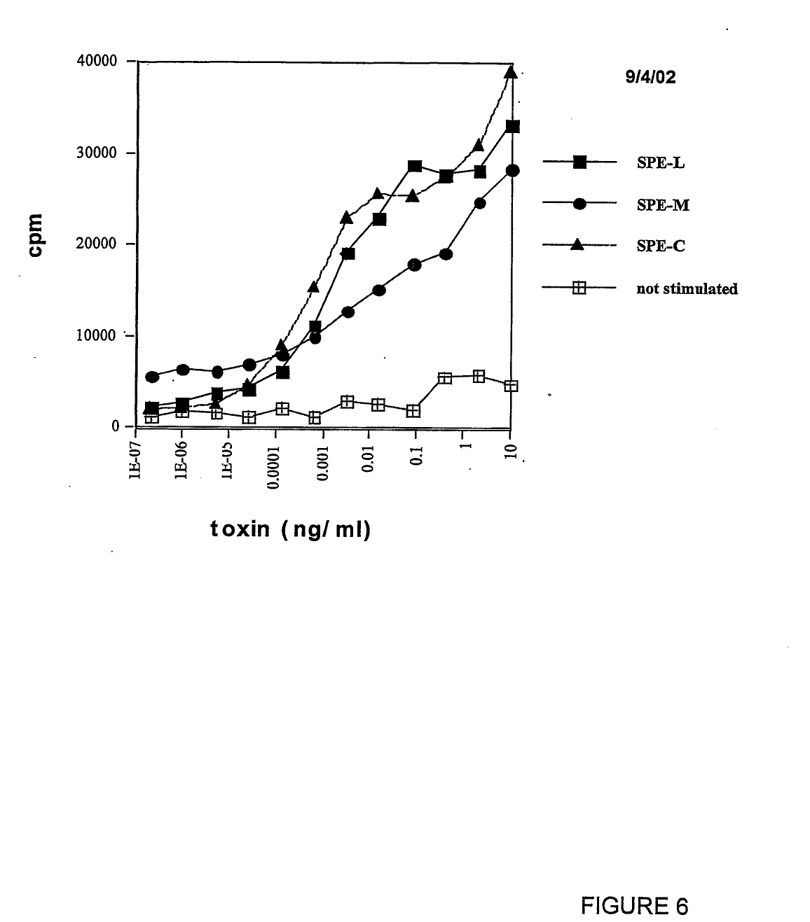

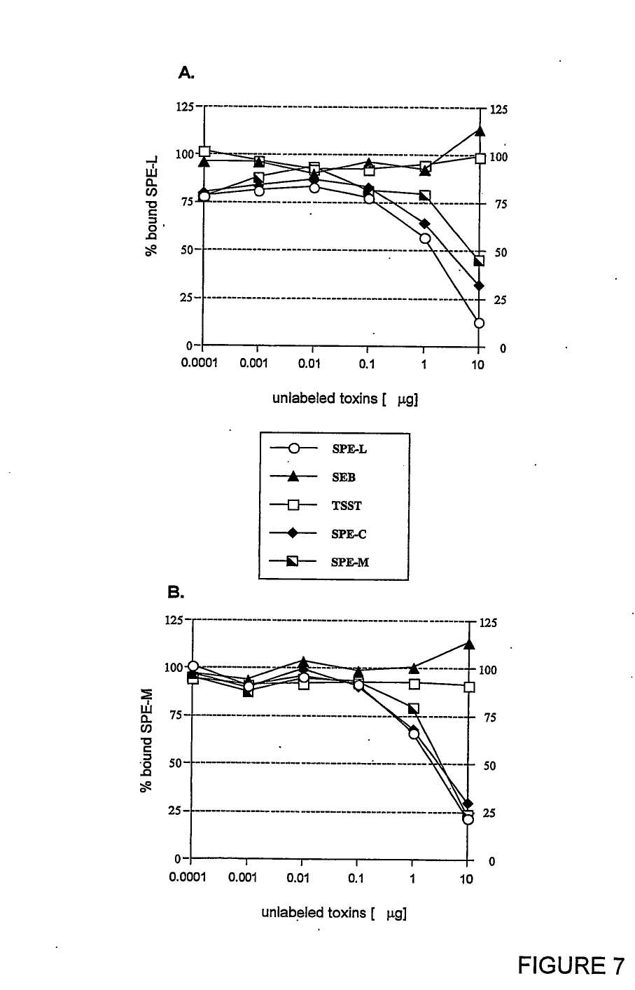

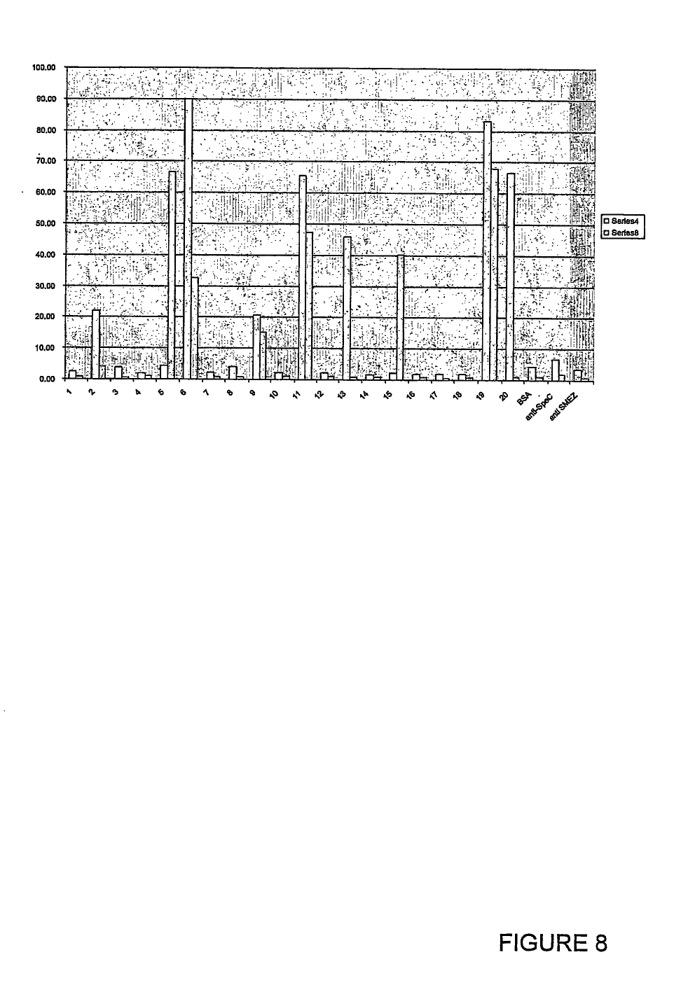

FIG. 4: The nucleotide sequence of the portion of the spe-m gene (SEQ ID NO. 3) coding the mature SPE-M superantigen (SEQ ID NO. 4). The gene was cloned from S. pyogenes M80 (isolate FP4223) using DNA primers designed from the DNA sequence of the orthologous sepe-m gene from S. equi. The DNA was analysed as described in FIG. 2.

FIG. 5: Family tree of streptococcal superantigens. The tree was created using the ClustalW server at http://clustalw.genome.ad.jp/ and the TreeView computer program (Page, R., 1996). The novel SAgs belong to the same subgroup as SPE-C, SPE-G, SPE-J and SMEZ, but build a separate branch within this subgroup.

FIG. 6: Stimulation of human T cells with recombinant toxins. Peripheral blood lymphocytes (PBLs) were isolated from human blood samples and incubated with varying concentrations of recombinant toxin (in duplicates). After 3d, 0.1 μCi [³H]-thymidine was added and cells were incubated for another 24 h, before harvested and counted on a gamma counter. [symbol] , unstimulated; ▴, rSPE-C; ▪, rSPE-L; ●, rSPE-M.

FIG. 7: Competition binding studies. LG-2 cells were incubated in duplicates with 1 ng of ¹²⁵I-labelled recombinant toxin and increasing amounts of unlabelled toxins. After 1 h cells were washed and counted.

A. Competition assay with labelled rSPE-L.

B. Competition assay with labelled rSPE-M.

♦, rSPE-C; ◯, rSPE-L; [symbol] , rSPE-M; ▴, SEB; □, TSST.

FIG. 8: Sero-conversion experiments. 20 sera from healthy human donors and 3 control samples (BSA, anti-SPE-C sera, anti-SMEZ sera) were incubated with 1 ng of ¹²⁵I-labelled rSPE-L and rSPE-M, respectively, for 1 h at 37 C. Protein A staphylococcal cells were added and incubated for 30 m on ice. The cells were then washed and counted. The Figure shows the percentage of bound toxin compared to the total amount of toxin used.

### DESCRIPTION OF THE INVENTION

The focus of the invention is the identification of two superantigens (SPE-L and SPE-M) and the corresponding polynucleotides which encode them.

The description is based on DNA sequence homology of sag genes, and in particular on short conserved peptide regions (PROSITE family signatures) in the deducted amino acid sequence. An open reading frame (ORF) for a hypothetical streptococcal pyrogenic exotoxin has been identified on the complete DNA sequence of prophage PhiHIH1.1 (NC003157) and labelled spe-1. The spe-1 gene has not been cloned before and no data describing the potential function of the SPE-L protein have been published.

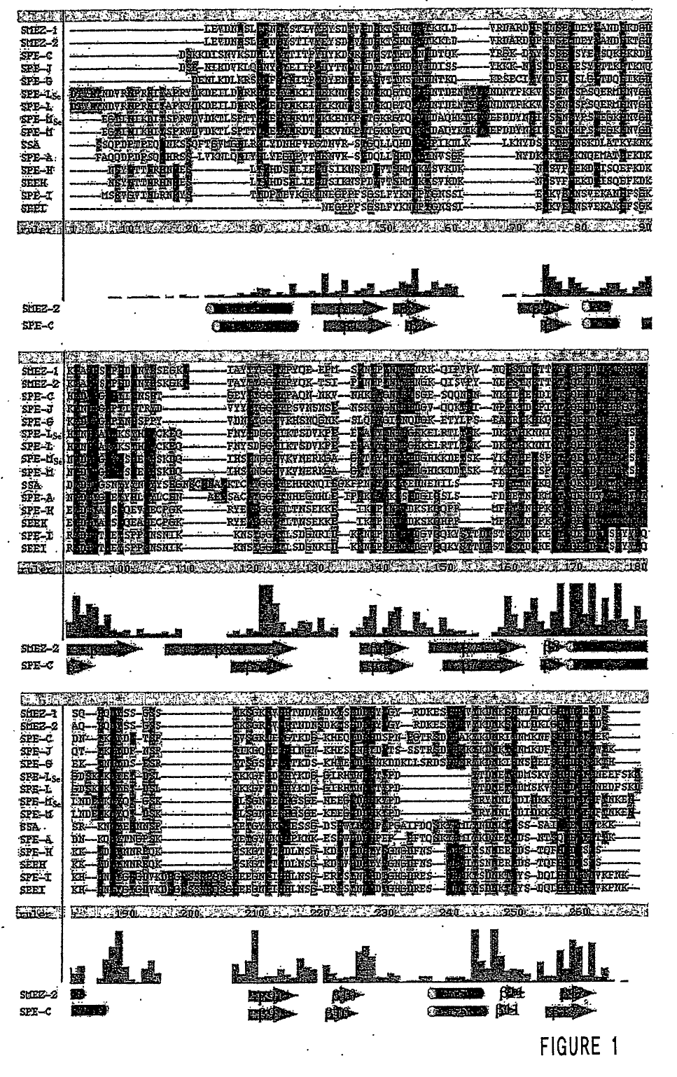

FIG. 1 shows the multiple alignment of amino acid sequences of SPE-L and SPE-M superantigens, the orthologous proteins SePE-L and SePE-M from S. equi and other streptococcal SAgs which have previously been identified in S. pyogenes and S. equi.

The applicant identified sepe-1 and sepe-m genes by mining the S. equi genomic data base at the Sanger Centre as mentioned herein after. Specific DNA primers were designed to amplify the genes coding for the mature SePE-L and SePE-M proteins from genomic S. equi DNA. The attempt to amplify sepe-1 and sepe-m from genomic DNA of 8 S. equi isolates failed suggesting that these genes are located on mobile DNA elements and occur in low frequencies in S. equi isolates.

The same primers were used to screen genomic DNA of 41 S. pyogenes isolates (including 29 M-types and 3 MNT) (FIG. 2A). Both genes were identified by the applicant, but in relatively low frequencies. The spe-1 gene occurred in a frequency of about 15% and was restricted to only 5 M-types (M28, M41, M56, M59, and M89). The M28 isolate was obtained from a patient with streptococcal toxic shock syndrome. M89 is known to be one of six M-types associated with acute rheumatic fever in New Zealand (Martin, D. et al., 1994). Genotyping of eleven M89 isolates from New Zealand (FIG. 2B) shows that the spe-1 gene occurs in relatively high frequency in that M-type (8 out of 11 carry the spe-1 gene).

The spe-m gene was found in a frequency of about 5% (the lowest ever reported for a streptococcal sag gene) in only 2 M-types (M80 and M92).

The amplified PCR products of spe-1 from isolate 10846 and spe-m from isolate FP4223 were cloned into pBlueScript vector and the DNA was analysed. FIG. 3 shows the nucleotide sequence (SEQ ID NO. 1) encoding mature SPE-L and the deducted amino acid sequence (SEQ ID NO. 2). FIG. 4 shows the nucleotide sequence (SEQ ID NO. 3) encoding mature SPE-M and the deducted amino acid sequence (SEQ ID NO. 4).

The genes for spe-1 and sepe-1 differ by only 7 base pairs and the deducted mature proteins by 4 amino acids. The genes for spe-m and sepe-m differ by 13 base pairs and the deducted mature protein by 8 amino acids.

Furthermore, most of the amino acid exchanges are conservative, suggesting that SPE-L/SePE-L and SPE-M/SePE-M have very similar, or the same, functions in the 2 streptococcal species and can be regarded as orthologues.

SPE-L and SPE-M show limited sequence homology to other streptococcal SAgs (≦30% sequence identity) and are most closely related to each other and to SPE-C. (FIG. 5). However, both protein sequences contain the characteristic staphylococcal enterotoxin/streptococcal pyrogenic exotoxin family signatures (PROSITE PS00277 and PS00278). Furthermore, SPE-L and SPE-M contain the C-terminal primary zinc binding motif (H-X-D), which is common among streptococcal SAgs and is a prerequisite for binding to the MHC class II β-chain (FIG. 1). The streptococcal SAg family tree (FIG. 5) shows that SPE-L and SPE-M belong to the ‘SPE-C subfamily’ (or Clade A), but build a separate branch within that group.

Significant differences in amino acid sequence between SPE-L/SPE-M and toxins of the SPE-C subfamily include an extended N-terminus (12-17 amino acids) and a 10 amino acid gap close to the C-terminus, which is unique among the whole SAg family (FIG. 1). In the SPE-C, SPE-H and SMEZ-2 protein structures, this region builds part of the β10-α5 loop and the as helix (Roussel, A. et al., 1997, Arcus et al., 2000). In contrast to loop regions, the structure determining regions, such as β-sheets and α-helices are generally well conserved in SAgs. The missing amino acids in this region suggest a major difference in the SPE-L/SPE-M protein structures compared to other SAgs.

The sequence homology of SPE-L and SPE-M are compared with other superantigens including SPE-C and SPE-G in Table 1.

In addition, while note wishing to be bound by any particular theory, the inventors' data on the TcR Vβ specificities of SPE-L and SPE-M indicate a different mode of action and function for SPE-L and SPE-M as compared superantigens known previously.

TABLE 1
Sequence Homology of Superantigens

| | SPE-C | SPE-G | SPE-J | SPE-L | SPE-M |
|---|---|---|---|---|---|
| SPE-L | 30%/40% | 26%/38% | 26%/38% | 100% | 42%/51% |
| SPE-M | 32%/40% | 26%/36% | 23%/36% | 42%/51% | 100% |

The inventors have identified that recombinant forms of SPE-L and SPE-M are extremely potent stimulators of human PBLs at nanomolar concentrations, confirming their role as SAgs. Sero-conversion against SPE-L and SPE-M was observed in several blood samples from healthy donors suggesting in-vivo expression of both toxins. Despite their highly conserved core structure, individual superantigens differ remarkably in their surface exposed regions. This explains why immune responses against SAgs are highly specific, e.g. polyclonal serum raised against a certain SAg does not cross-react with any other SAg (data not shown). Therefore, sero-conversion against a particular SAg is a strong indicator for the actual production of this SAg by the bacteria and also suggests a potential role as a virulence factor.

In accordance with the above, a first embodiment of the invention are the superantigens SPE-L and SPE-M having the amino acid sequence depicted in SEQ ID NO: 2 or SEQ ID NO: 4 or encoded by the nucleotide sequences depicted in SEQ ID NO:1 and SEQ ID NO: 3, respectively. In addition, the invention relates to the superantigens SePe-L and Sepe-M having amino acid sequences and/or being encoded by the sequence data provided herein.

In another embodiment, the invention represents the nucleic acids or polynucleotides depicted in SEQ ID NO:1 and SEQ ID NO:3, and their variants as referred to herein after.

The invention is of course not restricted to superantigens/polynucleotides having the specific sequences of SEQ ID NOS. 1 to 4. Instead, functionally equivalent variants are contemplated.

The phrase “functionally equivalent variants” recognises that it is possible to vary the amino acid/nucleotide sequence of a peptide while retaining substantially equivalent functionality. For example, a peptide can be considered a functional equivalent of another peptide for a specific function if the equivalent peptide is immunologically cross-reactive with and has at least substantially the same function as the original peptide. The equivalent can be, for example, a fragment of the peptide, a fusion of the peptide with another peptide or carrier, or a fusion of a fragment which additional amino acids. For example, it is possible to substitute amino acids in a sequence with equivalent amino acids using conventional techniques. Groups of amino acids normally held to be equivalent are:

(a) Ala, Ser, Thr, Pro, Gly;

(b) Asn, Asp, Glu, Gln;

(c) His, Arg, Lys;

(d) Met, Leu, Ile, Val; and

(e) Phe, Tyr, Trp.

Equally, nucleotide sequences encoding a particular product can vary significantly simply due to the degeneracy of the nucleic acid code.

Variants can have a greater or lesser degree of homology as between the variant amino acid/nucleotide sequence and the original.

Polynucleotide or polypeptide sequences may be aligned, and percentage of identical nucleotides in a specified region may be determined against another sequence, using computer algorithms that are publicly available. Two exemplary algorithms for aligning and identifying the similarity of polynucleotide sequences are the BLASTN and FASTA algorithms. The similarity of polypeptide sequences may be examined using the BLASTP algorithm. Both the BLASTN and BLASTP software are available on the NCBI anonymous FTP server of the NIH under /blast/executables/. The BLASTN algorithm version 2.0.4 [Feb. 24, 1998] set to the default parameters described in the documentation of variants according to the present invention. The use of the BLAST family of algorithms, including BLASTN and BLASTP, is described at NCBI's website under at URL /BLAST/newblast.html and in the publication of Altschul, Stephen F., et al. (1997), “Gapped BLAST and PSI-BLAST: a new generation of protein database search programs”, Nucleic Acids Res. 25:3389-34023. The computer algorithm FASTA is available on the Internet at the ftp site virginia.edu/pub/fasta/. Version 2.0u4, February 1996, set to the default parameters described in the documentation and distributed with the algorithm, is also preferred for use in the determination of variants according to the present invention. The use of the FASTA algorithm is described in W. R. Pearson and D. J. Lipman, “Improved Tools for Biological Sequence Analysis”, Proc. Natl. Acad. Sci. USA 85:2444-2448 (1988) and W. R. Pearson, “Rapid and Sensitive Sequence Comparison with FASTP and FASTA,” Methods in Enzymology 183:63-98 (1990).

The following running parameters provide a preferred example for determination of alignments and similarities using BLASTN that contribute to E values (as discussed below) and percentage identity. Persons of general skill in the art may appreciate alternative parameters. In addition, the the BLAST/FASTA default parameters may be used.

Unix running command: blastall -p blastn -d embldb -e 10-G 1-E 1 -r 2 -v 50 -b 50 -l queryseq -o results; and parameter default values:

-p Program Name [String]

-d Database [String]

-e Expectation value (E) [Real]

-G Cost to open a gap (zero invokes default behaviour) [Integer]

-E Cost to extend a cap (zero invokes default behaviour) [Integer]

-r Reward for a nucleotide match (blastn only) [Integer]

-v Number of one-line descriptions (V) [Integer]

-b Number of alignments to show (B) [Integer]

-i Query File [File In]

-o BLAST report Output File [File Out] Optional

For BLASTP the following running parameters are preferred: blastail -p blastp -d swissprotdb -e 10 -G 1 -E 1 -v 50 -b 50 -I queryseq -o results

-p Program Name [String]

-d Database [String]

-e Expectation value (E) [Real]

-G Cost to open a gap (zero invokes default behaviour) [Integer]

-E Cost to extend a cap (zero invokes default behaviour) [Integer]

-v Number of one-line descriptions (v) [Integer]

-b Number of alignments to show (b) [Integer]

-i Query File [File In]

-o BLAST report Output File [File Out] Optional

The “hits” to one or more database sequences by a queried sequence produced by BLASTN, BLASTP, FASTA, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN and FASTA algorithms also produce “Expect” or E values for alignments. The E value indicates the number of hits one can “expect” to see over a certain number of contiguous sequences by chance when searching a database of a certain size. The Expect value is used as a significance threshold for determining whether the hit to a database, such as the preferred EMBL database, indicates true similarity. For example, an E value of 0.1 assigned to a hit is interpreted as meaning that in a database of the size of the EMBL database, one might expect to see 0.1 matches over the aligned portion of the sequence with a similar score simply by chance. By this criterion, the aligned and matched portions of the sequences then have a 90% probability of being the same. For sequences having an E value of 0.01 or less over aligned and matched portions, the probability of finding a match by chance in the EMBL database is 1% or less using the BLASTN or FASTA algorithm.

According to one embodiment, “variant” polynucleotides, with reference to each of the polynucleotides of the present invention, preferably include sequences having the same number or fewer nucleic acids than each of the polynucleotides of the present invention and producing an E value of 0.01 or less when compared to the polynucleotide of the present invention. That is, a variant polynucleotide is any sequence that has at least a 99% probability of being the same as the polynucleotide of the present invention, measured as having an E value of 0.01 or less using the BIASTN or FASTA algorithms set at the parameters discussed above.

The term “variant polynucleotides” may include those which are complementary to, or hybridise to, the polynucleotides specifically exemplified herein.

Variant polynucleotide sequences will generally hybridize to the recited polynucleotide sequence under stringent conditions. As used herein, “stringent conditions” refers to prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

Polynucleotides of the invention may be used, for example, as primers and probes as is herein after detailed. Inasmuch as this is the case, it will be appreciated that “variant polynucleotides or nucleic acids”, in accordance with the invention, need not encode superantigens, or fragments thereof, which are functionally equivalent to SPE-L and/or SPE-M. It is enough that the polynucleotides may hybridise to the nucleic acids encoding SPE-L and/or SPE-M, or SePe-L or SePe-M, under stringent hybridisation conditions, or appropriate PCR conditions, for example.

It should be appreciated that polynucleotides encompassed by the invention may be DNA, RNA or cDNA for example, double stranded or single stranded, sense or antisense.

The superantigens of the invention together with their fragments and other variants may be generated by any one of a number of techniques standard in the art. For example, superantigens may be purified from natural sources such as S.pyogenes or S.equi. Alternatively, the superantigens and their variants or fragments may be synthetically or recombinantly produced. The inventors also contemplate production of a peptide of the invention by an appropriate transgenic animal.

It should be understood that in accordance with the invention, a superantigen of the invention, or variant or fragment thereof, is “isolated” or “purified”. An “isolated” or “purified” superantigen or fragment or variant thereof is one which has been identified and separated from the environment in which it naturally resides. It should be appreciated that ‘isolated’ does not reflect the extent to which they have been purified or separated from the environment in which it naturally resides.

Synthetic polypeptides having fewer than about 100 amino acids, and generally fewer than about 50 amino acids, may be generated by techniques well known to those of ordinary skill in the art. For example, such peptides may be synthesised using any of the commercially available solid-phase techniques such as the Merryfield solid phase synthesis method, where amino acids are sequentially added to a growing amino acid chain (see Merryfield, J. Am. Chem. Soc 85: 2146-2149 (1963)). Equipment for automative synthesis of peptides is commercially available from suppliers such as Perkin Elmer/Applied Biosystems, Inc. and may be operated according to the manufacturers instructions.

Recombinant means of producing the superantigens will also be appreciated by persons of general skill in the art to which the invention relates, having regard to the nucleic acid sequence information contained herein. In general terms, the superantigens may be produced recombinantly by inserting a polynucleotide (usually DNA) sequence that encodes the superantigen into an expression vector or construct (as detailed herein after) and expressing the superantigen in an appropriate host. Any of a variety of expression vectors known to those of ordinary skill in the art may be employed. Expression may be achieved in any appropriate host cell that has been transformed or transfected, by standard techniques, with an expression vector containing a DNA molecule which encodes the recombinant protein. Suitable host cells include prokaryotes, yeasts and higher eukaryotic cells. Preferably, the host cells employed are E. coli, yeasts or a mammalian cell line such as COS or CHO, or an insect cell line, such as SP9, using a baculovirus expression vector. The DNA sequence expressed in this matter may encode the naturally occurring superantigen, fragments of the naturally occurring protein or variants thereof, including the superantigen being expressed as a fusion protein.

A recombinant superantigen of the invention, or fragment or variant thereof, may be recovered from a transformed host cell, or culture media, following expression thereof using a variety of techniques standard in the art. For example, detergent extraction, osmotic shock treatment and inclusion body purification. The protein may be further purified using techniques such as affinity chromatography, ion exchange chromatography, filtration, electrophoresis, hydrophobic interaction chromatography, gel filtration chromatography, and chromatofocusing.

As mentioned herein before, in one embodiment, the invention provides nucleic acids or polynucleotides. These include those represented by SEQ ID NO: 1 and SEQ ID NO:2, and their variants. To the extent that a superantigen of the invention or variant or fragment thereof may be produced by recombinant techniques, it will be appreciated that the invention also provides nucleic acid constructs comprising nucleic acids encompassed by the invention.

Those of general skill in the art to which the invention relates will readily be able to identify nucleic acids applicable to the invention. This may be done on the basis of the nucleic acid and amino acid sequences herein, the genetic code, the understood degeneracy therein, and the information further provided herein.

DNA sequences encoding the superantigens, functional equivalents or fragments may be obtained, for example, by screening an appropriate S. pyogenes cDNA or genomic DNA library for DNA sequences that hybridise to degenerate oligonucleotides derived from partial amino acid sequences of the superantigen. Suitable degenerate oligonucleotides may be designed and synthesised by standard techniques and the screen may be performed as described, for example, in Maniatis et al. Molecular Cloning—A Laboratory Manual, Cold Spring Harbour Laboratories, Cold Spring Harbour, N.Y. (1989).

It should be understood that a nucleic acid in accordance with the invention, is an “isolated” or “purified” nucleic acid. An “isolated” or “purified” nucleic is one which has been identified and separated from the environment in which it naturally resides. It should be appreciated that ‘isolated’ does not reflect the extent to which the nucleic has been purified or separated from the environment in which it naturally resides. Isolated or purified nucleic acids in accordance with the invention may be purified from natural sources, or preferably derived by chemical synthesis or recombinant techniques. Such techniques are readily known in the art to which the invention relates.

Nucleic acid constructs in accordance with the invention will generally contain heterologous nucleic acid sequences; that is nucleic acid sequences that are not naturally found adjacent to the nucleic acid sequences of the invention. The constructs or vectors may be either RNA or DNA, either prokaryotic or eukaryotic, and typically are viruses or a plasmid. Suitable constructs are preferably adapted to deliver a nucleic acid of the invention into a host cell and are capable of replicating in such cell. Recombinant constructs comprising nucleic acids of the invention may be used, for example, in the cloning, sequencing, and expression of nucleic acid sequences of the invention.

Those of skill in the art to which the invention relates will recognise many constructs suitable for use in the present invention.

A recombinant construct or vector comprising a nucleic acid molecule of the invention may be generated via recombinant techniques readily known to those of ordinary skill in the art to which the invention relates.

In the case of expression constructs, the inventors contemplate the use in the present invention of vectors containing regulatory sequences such as promoters, operators, repressors, enhancers, termination sequences, origins of replication, and other appropriate regulatory sequences as are known in the art. Further, the vectors may contain secretory sequences to enable an expressed protein to be secreted from its host cell. In addition, the expression vectors may contain fusion sequences (such as those that encode a heterologous amino acid motif, for example Ubiquitin) which lead to the expression of inserted nucleic acid sequences of the invention as fusion proteins or peptides.

Identification of these superantigens and of their properties by the applicant gives rise to a number of useful applications.

A first application is diagnostic. As used herein, the term “diagnostic” should be taken in a broad context. Diagnostic applications include, for example, genotyping of organisms by reference to their superantigen profile, subtyping of strains of S. pyogenes, identification of specific M-types of S. pyogenes, and subtyping of specific M-types of S. pyogenes (for example as SPE-L or SPE-M positive or negative). Such diagnostic procedures may be conducted on laboratory samples, or samples taken from subjects, which could aid in the diagnosis of infection of a subject by a particular M-type of S. pyogenes.

As mentioned SPE-L and SPE-M occur in relatively low frequencies (15% and 5% respectively). Both superantigens appear to be restricted only to certain S. pyogenes M-types: in the case of SPE-L M28, M41, M56, M59 and M89; and, in the case of SPE-M, M80 and M92. This provides for rapid diagnostic testing to identify S. pyogenes isolates, or at least the presence of the superantigens of the invention, or functional equivalents thereof. This may have advantages in early identification of the nature of the superantigens, or bacterial isolates responsible for infection of one or more subjects. Such testing may allow for the study of transmission of infection, tracking of outbreaks or epidemics, and ultimately facilitate early treatment of infection, allow for focused isolate- or superantigen-specific treatment methods, and also containment.

S. equi is a Lancefield group C. Streptococcus which causes strangles (a contagious inflammatory disease of the upper respiratory tract and associated lymph nodes) of equids. Features include high fever, neutrophilia and fibrinogenemia. Cases of infections of humans with S. equi have been reported. This invention also provides a method of identifying S. equi infection based on the similarity between SePE-L/SPE-L and SePE-M/SPE-M. This may have the advantages and applications as mentioned in the immediately preceding paragraph, for example.

In addition to the above mentioned diagnostic applications, such methods may be used to ensure integrity of laboratory cultures of at least the S.pyogenes M-types mentioned above, or S.equi, or contamination of laboratory cultures by these bacteria.

The general diagnostic approaches applicable to the invention may be achieved by various techniques, for example nucleic acid hybridisation (Southern hybridisation for example), targeted nucleic acid amplification (for example, PCR), or by using antibodies specific to the superantigens of the invention.

Having the information provided herein, and knowledge of standard technical procedures, persons skilled in the art will appreciate means of realising such diagnostic procedures.

However, by way of example, a sample can be taken from a laboratory culture, or a subject, and processed to isolate either the superantigen of interest, or nucleic acid, from the bacterial population present in the sample. Suitable extraction and purification procedures are known in the art. However, by way of example see Proft et al, 2000, or Gerlach et al, 2000.

As used herein, a “subject” may be any animal which may be at risk of, or has been, infected with a bacteria particularly S.pyogenes, or S.equi) expressing a superantigen of the invention, or functional equivalent thereof. Most preferably, the subject is human. Alternatively, the subject is an equid.

Where the method of this aspect of the invention involves testing a “sample” from a subject for the presence or absence of a superantigen of the invention the sample may be any suitable body tissue or fluid. Most preferably, the sample is saliva or blood.

Detection of the presence of a superantigen of the invention, or functional equivalents thereof, or determination of the subtype of S.pyogenes or S.equi may be achieved by providing a set of primers which amplify either all or a subset of superantigen genes and that generate gene specific fragments. This can be modified to provide a simple qualitative ELISA-strip type kit that detects biotin labelled PCR fragments amplified by the specific primers and hybridised to immobilised sequence specific probes.

According to the invention, a primer is an oligonucleotide capable of specific hybridisation under particular PCR conditions to a region of the template DNA, which has a sequence which is substantially complementary to the primer sequence, and is adapted to prime the extension of DNA during PCR. It will be realised that a complementary sequence is capable of forming Watson-Crick bonds with its complement, in which adenine pairs with thymine or guanine pairs with cytosine. Each primer is typically used as a member of a primer pair, including a 5′ upstream primer that hybridises with the 5′ end of the template DNA to be amplified and a 3′ downstream primer that hybridises with the complement of the 3′ end of the template DNA to be amplified.

Those of ordinary skill in the art to which the invention relates will understand that the term “substantially complementary”, as used herein, means that the primer may not have 100% complementarity to its target template sequence but is still capable of annealing thereto in a specific manner under appropriate PCR annealing conditions.

The primers of the present invention may be prepared by any number of conventional DNA synthesis methods.

The nucleotide sequence information provided herein may be used to design primers for amplification of parts of the spe-1 or spe-m genes. An oligonucleotide for use in probing or PCR may be about 30 or fewer nucleotides in length. Generally, specific primers are upwards of 14 nucleotides in length. For optimum specificity and cost effectiveness, primers or 16-24 nucleotides in length are preferred. Those skilled in the art are well versed in the design of primers for use in processes such as PCR.

Primers of particular use in this embodiment of the invention include sepe-1.fw, sepe-1.rev, sepe-m.fw and sem.rev, the sequences of which are described herein after in the Examples section as well in the sequence listing (as SEQ ID NO'S. 5, 6, 7 and 8, respectively).

In addition, the inventors believe the following primers find use in this embodiment of the invention:

(referred to as “Spe-1 forward 2”, SEQ ID NO: 9)

GATACGTACAATACAAATG -

(referred to as “Spe-1 reverse 2”, SEQ ID NO: 10)

AATAGCATTCGACC -

(referred to as “Spe-m forward 2”, SEQ ID NO: 11)

GAGGGGACTATTAATATTAAG -

(referred to as “Spe-m reverse 2”, SEQ ID NO: 12)

GGTTTCTTGATACTAAC -.

In accordance with the preferred embodiment, the inventors believe optimal results may be obtained using primers which are identical in length and sequence to those above mentioned (ie SEQ ID NOS 5 to 12). However, a person of ordinary skill in the art will recognise that alterations may be made to the primers while still maintaining the specificity of the PCR amplification and the efficacy of the present inventive diagnostic method. For example, the length of the primers used may be varied, including the addition of non-complementary nucleotide fragments being attached to the 5′ end of the primers. In addition, minor changes (or conservative alterations) to the sequence of the primers which do not substantially alter their ability to anneal to their specific target DNA and subsequently prime extension during PCR, may be made. For example, any particular nucleotide, or plurality of nucleotides, of a primer may be substituted for alternative nucleotides, which may not allow for Watson-Crick base-pairing at the particular site of alteration on annealing of the primer to the template DNA during PCR, but nonetheless does not substantially affect the ability of the primer to prime extension during PCR.

The inventors contemplate the design of primers which map to any portion of the superantigen genes defined herein. However, the inventors note the nucleic acid sequence in the regions ncl 1-270, 295-335, 350-380, 410-440, 560-620, 643-657 of Spe-1 and regions ncl 1-265, 280-323, 333-370, 390-430 and 520-595 of Spe-m may provide good targets for primer design due to their low degree of homology with superantigen family members. Primers designed to these regions of the genes may allow for specificity.

It will be appreciated that the usefulness of primer sets other than those specifically identified above may be evaluated, at least notionally, using appropriate software and the DNA sequence information herein. Software packages of use in primer design include, for example, PC Oligo5 (National Bioscience Inc) or Amplify (University of Wisconsin).

Generally, only one PCR, using a single primer set, will be needed in order to identify the M types of S.pyogenes or S.equi, or the presence or a superantigen of the invention within a sample. However, it will be appreciated that there may be times where a parallel PCR, using the second primer set, may be utilised in order to further clarify the identity of a species present within a sample. Similarly, upon optimisation of the PCR conditions both novel primer sets may be used in a single PCR.

PCR products may be detected using standard methodology, for example gel electrophoresis. In addition, the PCR products may labelled to aid detection. Such labelling may include, for example, end-labelling of the primers with [γ-³³P]ATP. Alternatively, other means of labelling the PCR products may be utilised; for example, incorporation of [α-³²P]dNTPs during PCR amplification, or, non-radioactive labelling systems using digoxygenin, biotin and the like, may be employed.

Each PCR is run with at least one monospecific control sample or standard of known species identity. It will be appreciated that control samples containing more than one known species of S.pyogenes or S.equi or specific superantigens of the invention, may be entertained. Negative controls in which no template DNA of S.pyogenes or S.equi, or specific superantigens of the invention, is present may also be run against the samples. It will be appreciated that other standard controls routinely used in the art may also be used.

Amplification is conducted according to conventional procedures in the art to which this invention relates; for example, see PCR Primer: a laboratory manual, eds CW Dieffenbach, GS Dveksler (CSHL Press). However, in general terms, PCR according to the invention may include approximately 20 to 100 ng of template DNA, approximately 0.1 μM-1 μM of each primer, approximately 100-200 μM each dNTP, approximately 1-7 mM MgCl₂, and approximately 0.5-1 U Taq DNA polymerase. More preferably the PCR is run using approximately 50 ng of template DNA, approximately 1 μM of each primer, approximately 200 μM each dNTP, approximately 2-4 mM MgCl₂, and approximately 1 U Taq DNA polymerase.

Typically, each PCR is overlayed with mineral oil or the like to prevent evaporation of the reaction mix during cycling. PCR cycling according to the invention may include the following conditions: denaturation at a temperature of 94° C. for 30 to 60 seconds, annealing at a temperature of from 45° C. to 60° C. for 30 to 60 seconds and extension at a temperature of 72° C. for 30 to 60 seconds. Between 30 and 40 cycles may be run. More preferably, PCR cycling includes the following conditions: denaturation at a temperature of 94° C. for 30 seconds, annealing at a temperature of from 45° C. to 60° C. for 30 to 60 seconds, extension at a temperature of 72° C. for 30, with 30 to 35 cycles run.

In the case of the use of the primers SEQ ID NOS 5 and 6, or 7 and 8, the inventors have found the conditions provided herein after under the heading “Identification of novel SAGs and Genotyping of S.equi and S.pyogenes isolates” within the Examples section, to be particularly useful.

It will be appreciated by those of ordinary skill in the art that the PCR conditions provided herein are merely exemplary and may be varied so as to optimise conditions where, for example, alternative PCR cyclers or DNA polymerases are used, where the quality of the template DNA differs, or where variations of the primers not specifically exemplified herein are used, without departing from the scope of the present invention. The PCR conditions may be altered or optimised by changing the concentration of the various constituents within the reaction and/or changing the constituents of the reaction, altering the number of amplification cycles, the denaturation, annealing or extension times or temperatures, or the quantity of template DNA, for example. Those of skill in the art will appreciate there are a number of other ways in which PCR conditions may be optimised to overcome variability between reactions.

It will be understood that where not specifically exemplified herein appropriate PCR annealing temperatures for any primer within the scope of the present invention may be derived from the calculated melting temperature of that primer. Such melting temperatures may be calculated using standard formulas, such as that described in Sambrook, 1989 (Sambrook, Fritsch, Maniatis. Molecular loning—Laboratory Manual, Volume 2, 11.45-11.57). As will be understood by those of ordinary skill in the art to which this invention relates annealing temperatures may be above or below the melting temperature but generally an annealing temperature of approximately 5° C. above the calculated melting temperature of the primer may be suitable.

While the novel primers above mentioned have been designed to enable specific PCR amplification of regions of the superantigen genes of the invention, it will be appreciated that they may also be applied, individually or in combination, to various other applications. For example they may be used as molecular probes, or primers for alternative diagnostic techniques (such as LCR, ligase chain reaction).

As mentioned herein before, diagnostic methods of the invention may employ probing using for example the Southern blotting technique. Persons skilled in the art will readily appreciate procedures for Southern blotting, having regard to the nucleic acid sequence information herein, and standard protocols used in the art (as described for example in Sambrook, 1989). However, by way of general example, DNA may be extracted from a sample and digested with different restriction enzymes. Restriction fragments may then be separated by electrophoresis on an agarose gel, before denaturation and transfer to a nitrocellulose filter. Labelled probes may be hybridised to the DNA fragments on the filter and binding determined. DNA for probing may be prepared from RNA preparations from cells. Probing may optionally be done by means of so-called “nucleic acid chips” (see Marshall and Hodgson (1998) Nature Biotechnology 16:27-31).

Probes for use in this embodiment of the invention include any oligonucleotide capable of hybridising to at least a portion of any one of the genes of the specific superantigens of the invention. Such probes include any of the primers herein before described. In addition, nucleic acids representing the entire gene sequence of a superantigen of the invention may be used.

It should be appreciated that probes of the invention include both those nucleic acids which have 100% complementarity to the region of the target gene they are to hybridise to, as well as those nucleic acids which do not show 100% complementarity but are capable of hybridising to the target under stringent hybridisation conditions (herein before detailed) to ensure specificity of the diagnostic method.

Probes or primers in accordance with the invention may be derived by any means commonly used in the art; for example, chemical synthesis, restriction digestion of a recombinant vector and subsequent isolation of the desired oligonucleotide.

Another diagnostic approach is to provide monoclonal antibodies to detect each of the streptococcal superantigens. An ELISA kit containing such antibodies would allow the screening of large numbers of streptococcal isolates. A kit such as this would be useful for agencies testing for patterns in streptococcal disease or food poisoning outbreaks.

Antibodies to the superantigens for use in applications such as are described above are also provided by this invention. Such antibodies can be polyclonal but will preferably be monoclonal antibodies.

Monoclonal antibodies with affinities of 10⁻⁸ M⁻¹ or preferably 10⁻⁹ to 10⁻¹⁰ M⁻¹ or stronger will typically be made by standard procedures as described, eg. in Harlow & Lane (1988) or Goding (1986). Briefly, appropriate animals will be selected and the desired immunization protocol followed. After the appropriate period of time, the spleens of such animals are excised and individual spleen cells fused, typically, to immortalised myeloma cells under appropriate selection conditions. Thereafter, the cells are clonally separated and the supernatants of each clone tested for their production of an appropriate antibody specific for the desired region of the antigen.

Other suitable techniques for preparing antibodies well known in the art involve in vitro exposure of lymphocytes to the antigenic polypeptides, or alternatively, to selection of libraries of antibodies in phage or similar vectors.

Also, recombinant immunoglobulins may be produced using procedures known in the art (see, for example, U.S. Pat. No. 4,816,567 and Hodgson J. (1991)).

The antibodies may be used with or without modification. Frequently, antibodies will be labelled by joining, either covalently or non-covalently, a substance which provides for a detectable signal. A wide variety of labels and conjugation techniques are known and are reported extensively in the literature. Suitable labels include radionuclides, enzymes, substrates, cofactors, inhibitors, fluorescent agents, chemiluminescent agents, magnetic particles and the like. Patents teaching the use of such labels include U.S. Pat. Nos. 3,817,837; 3,850,752; 3,939,350; 3,996,345; 4,277,437; 4,275,149; and 4,366,241.

The immunological assay in which the antibodies are employed can involve any convenient format known in the art.

In another embodiment the invention provides another diagnostic method which allows for the determination of whether a subject has been exposed to a superantigen, or specific S.pyogenes or S.equi bacteria expressing same. This method relies on the fact that a subject will develop neutralising antibodies to the superantigen following infection. This method has application in the study of transmission of disease and infections based on S.pyogenes or S.equi. In addition, it has application in determination of individuals who may be susceptible to, or those immune to, infection or ultimate illness due to infection, during epidemics and the like. This may allow for more targeted vaccination and treatment protocols.

In general terms, this embodiment of the invention involves the study of a sample of peripheral blood lymphocytes (PBLs) from a subject to be tested using superantigens of the invention or functionally equivalent variants or fragments thereof. The peripheral blood lymphocytes are prepared from a blood sample of the subject. Superantigen induced T cell proliferation with the test sample compared to a control provides an indication of the presence or absence of neutralising antibodies to the superantigens. A low level of T cell proliferation compared to the control indicates the presence of neutralising antibodies against the superantigens used in the assay. A level of T cell proliferation equal to or higher than that of the control, is indicative of the absence of neutralising antibodies.

Having regard to the information herein, persons of general skill in the art to which the invention relates will readily appreciate methods for conducting this embodiment of the invention. However, by way of example, PBLs are purified from a subject to be tested and stimulated as described herein after under the heading “Toxin Proliferation Assay”, with the exception that 10% fetal calf serum is replaced with 5% FCS plus 5% subject serum. Recombinant superantigens are preferably used at sub-saturating concentrations. Neutralising response is determined by comparing the T cell proliferation with a control (10% FCS). Relative inhibition may be calculated as 1-cpm (patient serum)/cmp (FCS).

In addition to diagnostic applications, another application of the superantigens is reliant upon their ability to bind to other cells.

One of the most important features of superantigens is that they bind a large number or T cell receptor molecules by binding to the Vβ domain. They are the most potent of all T cell mitogens and are therefore useful to recruit and activate T cells in a relatively non-specific fashion.

This ability enables the formation of constructs in which the superantigen (or at least the T-cell binding portion of it) is coupled to a cell-targeting molecule, particularly an antibody, more usually a monoclonal antibody.

When a monoclonal antibody that targets a specific cell surface antigen (such as a tumor specific antigen) is coupled to a superantigen in such a construct, this generates a reagent that on the one hand will bind specifically to the tumor cell, and on the other hand recruit and selectively active T cells for the purpose of killing the targeted cell.

Bi-specific constructs of this type have important applications in therapy (particularly cancer therapy) and again may be prepared by means known to those skilled in art. For example SPE-M may be coupled to a tumor specific monoclonal antibody. The constructs may be incorporated into conventional carriers for pharmaceutically-active proteins.

In addition, the superantigens of the invention may be used to form immunomodulatory constructs in accordance with the techniques outlined in PCT/NZ01/00267.

In another embodiment, the invention relates to kits for the detection of superantigens SPE-L or SPE-M, or functionally equivalent variants thereor, or nucleic acids encoding same. Such kits may generally comprise at least an antibody directed to SPE-L, SPE-M or functionally equivalent variants thereof. Alternatively, or in addition, such kits may comprise at least one nucleic acid adapted to hybridise to a nucleic acid encoding SPE-L, SPE-M or a functionally equivalent variant thereof. It will be appreciated such kits will find application in the diagnostic methods described herein before.

Various aspects of the invention will now be described with reference to the following experimental section, which is included for illustrative purposes.

### EXAMPLES

Section A: Superantigen Identification and Characterization

Materials and Methods

Identification of novel SAGs and Genotyping of S.equi and S.pyogenes isolates The novel superantigens were identified by screening a panel of group A Streptococci (GAS) isolates using primers designed from the DNA sequences of the S. equi orthologous genes sepe-1 and sepe-m, respectively.

The New Zealand S. pyogenes isolates were obtained from the Institute of Environmental Sciences and Research in Porirua. Isolate 33117 was obtained from Pasteur Institute, Lille, France. The S. equi isolates were obtained from the Department of Microbiology, Otago University, Dunedin.

The streptococcal isolates were grown in Brain Heart Infusion medium (BHI, Difco) at 37 C and genomic DNA was prepared as described previously (Proft et al, 2000).

The screening or genotyping was carried out by PCR with 50 ng of purified streptococcal genomic DNA using the specific primer pairs:

sepe-1.fw (GCGGATCCGATACGTACAATACAAATG), and

sepe-1.rev (GCGAATTCAATAGCATTCGACC); and,

sepe-m.fw (GCGGATCCGAGGGGACTATTAATATTAAG), and

sem.rev (GCGAATTCGGTTTCTTGATACTAAC).

In addition, a primer pair specific to a DNA region encoding the 23S rRNA, oligo 23rRNA fw (GCTATTTCGGAGAGAACCAG) and oligo 23rRNA rev (CTGAAACATCTAAGTAGCTG) was designed and used for PCR as a positive control [Proft et al, 1999].

The primers above were manufactured by Sigma, Australia. PCR was carried out in 0.5 ml PCR tubes (SSI, Ca, USA) with 50 ng of template DNA, 0.2 μM of each primer, 100 μM of each dNTP (GibcoBRL), 2.5 mM MgCl₂, and 1 U Taq DNA polymerase (Promega). Each PCR was overlayed with mineral oil or the like to prevent evaporation of the reaction mix during cycling. PCR cycling was run under the following conditions using a Perkin Elmer Cetus Thermo Cycler: denaturation at a temperature of 94° C. for 30 seconds, annealing at a temperature of from 53° C. for 45 seconds and extension at a temperature of 72° C. for 30 seconds. 30 cycles were run.

Sepe-1 and sepe-m were identified by searching the microbial genome databases at the National Center for Biotechnology Information, including the S. equi genome database contributed by the Sanger Centre (database Taxonomy ID 1336). These databases are available online at the web site of the National Center for Biotechnology Information. The searches were conduced using highly conserved β5 and a4regions of streptococcal and staphylococcal superantigens, using a TBLASTN search program available through the BLAST facility on the web site.

The ORFs were defined by translating the DNA sequences around the matching regions and aligning the protein sequences to known superantigens using the computer programs Clustal W (Thompson et al, 1994] which can be accessed online at GenomeNet, a web site of Kyoto University. Multiple alignments and dendrograms were performed with ClustalX. The FASTA programme was used for searching the SwissProt (Amos Bairoch, Switzerland) and PIR (Protein Identification Resource, USA) protein databases.

The leader sequences of SePE-L and SePE-M were predicted using the SignalP server, available in the CBS Prediction Servers section of the web site of the Centre for Biological Sequence Analysis (Technical University of Denmark DTU) (Nielsen et al, 1997).

Cloning of spe-1 and spe-m.

Fifty nanograms of Spyogenes M89 (isolate 10846) or Spyogenes M80 (isolate FP4223) genomic DNA was used as a template to amplify the spe-1 DNA fragment and the spe-m DNA fragment, respectively, by PCR using the primer pairs sepe-1.fw/sepe-1.rev and sepe-m.fw/sepe-m.rev, respectively (see above).

The primers contain a terminal tag with the restriction enzyme recognition sequences BamHI and EcoRI, respectively. The amplified DNA fragment, encoding the mature protein without the predicted leader sequences (Kamezawa et al, 1997 Infect. Immun. 65 no9:38281-33) were enriched using the Wizard PCR DNA purification system (Promega) and cloned into T-tailed pBlueScript SKII vectors (Stratagene), followed by transformation into E.coli XL1 blue.

The DNA sequences of the subcloned toxin genes were confirmed by the dideoxy chain termination method using a Licor automated DNA sequencer (model 4200). As the DNA sequences from the genomic database are all unedited raw data, 3 subclones of every cloning experiment were analyzed to ensure that no Taq polymerase related mutations were introduced.

The DNA sequences have been annotated in EMBL/Genbank/DDBJ. The accession numbers are AF514282 for spe-l and AF514283 for spe-m.

Expression and purification of recombinant SPE-L and rSPE-M.

Subcloned spe-1 and spe-m fragments were cut from pBlueScript SKII vectors, using restriction enzymes BamHI and EcoRI (LifeTech), and cloned into pET32-3c expression vector. This vector is a modified version of pET32a (Novagene) that expresses the highly specific protease 3C cleavage site from a picornavirus (Walker et al, 1994.) just upstream of the inserted DNA. Recombinant SPE-M and rSPE-L were expressed in E. coli AD494 (DE3) (Novagene) as thioredoxin fusion proteins. Cultures were grown at 30 degrees C. and induced for 3-4 h after adding 0.2 mM isopropyl-β-D-thiogalactopyranoside (IPTG).

The thioredoxin (Trx) fusion proteins were purified on Ni²⁺ IDA sepharose (Sigma) and the mature proteins were cleaved off from thioredoxin on the column using protease 3C (provided by Keith Hudson, University of Oxford, Oxford, UK) overnight at room temperature. The eluates containing the recombinant toxins were collected.

Gel Electrophoresis

All purified recombinant toxins were tested for purity on a 12.5% SDS-polyacrylamide gel according the procedure of Laemmli.

Toxin Proliferation Assay

Human peripheral blood lymphocytes (PBL) were purified from blood of a healthy donor by Histopaque Ficoll (Sigma) fractionation. The PBL were incubated in 96-well round bottom microtiter plates at 10⁵ cells per well with RPMI-10 (RPMI with 10% fetal calf serum) containing varying dilutions of recombinant toxins. The dilution series was performed in 1:5 steps from a starting concentration of 10 ng/ml of toxin. Pipette tips were changed after each dilution step. After 3 days 0.1 μCi [³H]thymidine was added to each well and cells were incubated for another 24 h. Cells were harvested and counted on a scintillation counter.

TcR Vβ Analysis

Vβ enrichment analysis was performed by anchored multiprimer amplification (Hudson et al, 1993, J exp Med 177:175-185). Human PBLs were incubated with 20 pg/ml of recombinant toxin at 10⁶ cells/ml for 3 d. A two-fold volume expansion of the culture followed with medium containing 20 ng/ml IL-2. After another 24 h, stimulated and resting cells were harvested and RNA was prepared using Trizol reagent (Life Tech). A 500 base pairs β-chain DNA probe was obtained by anchored multiprimer PCR as described previously (Hudson et al 1993), radiolabelled and hybridized to a panel of 20 individual Vβs and a Cβ DNA region dot blotted on a Nylon membrane. The membrane was analysed on a Molecular Dynamics Storm Phosphor imager using ImageQuant software. Individual Vβs were expressed as a percentage of all the Vβs determined by hybridization to the Cβ probe.

Radiolabelling and LG2 Binding Experiments

Recombinant toxin was radioiodinated by the chloramine T method as previously described (by Li et al. 1997). Labelled toxin was separated from free iodine by size exclusion chromatography using Sephadex G25 (Pharmacia). LG2 cells were used for cell binding experiments, as described (Li et al. 1997). Briefly, cells were harvested, resuspended in RPMI-10 and mixed at 10⁶ cells/ml with ¹²⁵I-tracer toxin (1 ng) and 0.0001 to 10 μg of unlabelled toxin and incubated at 37° C. for 1 h. After washing with ice cold RPMI-1 the pelleted cells were analyzed in a gamma counter.

For competitive binding studies, 1 ng of ¹²⁵I-tracer toxin (rSPE-L or rSPE-M) was incubated with 0.0001 to 10 μg of unlabeled toxin (rSPE-L, rSPE-M, rSPE-C, rSEB, and rTSST) for 1 h.

Seroconversion Experiments:

5 microlitres of serum from human healthy donors were mixed with 10 μl of PBS and 10 μl of ¹²⁵I-labelled recombinant SPE-L or rSPE-M and incubated for 1 h at 37 degrees C. As controls, BSA or rabbit antiserum against SPE-C or SMEZ was used instead of human sera. After adding 50 μl of protein A—staphylococcal cells (Toxin Technology) the samples were incubated for another 30 min at 4 degrees C. before the cells were spun down, washed in 1 ml of PBS and counted in a Cobra Gamma Counter.

Results

Identification and Sequence Analysis of Superantigens.

The Sanger Centre Streptococcus equi genome database is accessible via the internet and contains a collection of more than 200 DNA sequence contigs derived from a shot gun plasmid library of the complete S. equi genome. This database was searched with a highly conserved superantigen peptide sequence, using a search program that screens the DNA database for peptide sequences in all 6 possible reading frames. 4 significant matches and predicted ORFs with significant homology to streptococcal and staphylococcal superantigens were found by aligning translated DNA sequences to complete protein sequences of known SAgs.

Two of these ORFs correlate to the previously described SePE-I and SePE-H (Artiushin et al., 2002). The third ORF identified showed strong sequence homology to the hypothetical exotoxin gene spe-1 found on prophage PhiHIH 1.1 sequence (NC003157). This was labelled sepe-1 according to the S. equi nomenclature (Streptococcus equi pyrogenic exotoxin 1). The fourth ORF showed no significant sequence homology to any known superantigen, but contains the 2 staphylococcal enterotoxin/streptococcal exotoxin family signatures PS00277 and PS00278 (Prosite database, http://www.expasy.ch/prosite/) and was named sepe-m. (see FIGS. 4 and 5).

Genotyping of S. equi and S. pyogenes Isolates

Genotyping of 8 S. equi isolates obtained from Otago University (Dunedin) using specific primer pairs for the sepe-1 and sepe-m genes failed to detect the novel sag genes, suggesting that they are located on mobile DNA elements and restricted to certain S. equi isolates and thus restricted in their distribution to certain S.equi isolates.

Screening of S. pyogenes isolates (including 29 M-types and 3 MNT) revealed the sepe-1 gene in 6/40 cases (15%) and the sepe-m gene in 2/40 cases (5%) FIG. 2. The sepe-1 gene was restricted to only 5 M-types (M28, M41, M56, M59 and M89) and the sepe-m gene was found in only 2 M-types (M80 and M92).

Interestingly, the M28 isolate was obtained from a patient suffering from streptococcal toxic shock syndrome and M89 strains are strongly associated with acute rheumatic fever in New Zealand (Martin, D. et al., 1994).

In addition, 11 S. pyogenes M89 isolates from New Zealand were genotyped and the spe-1 gene was found in 8 isolates indicating a significant link between this M-type and the spe-1 gene (FIG. 2B).

Cloning and DNA Analysis of spe-1 and spe-m from S. pyogenes

The sepe-1 gene was cloned from isolate 10846 (M89) and DNA sequence analysis revealed a strong sequence homology to the S. equi sepe-1. The gene was named spe-1 according to the S. pyogenes nomenclature for the streptococcal pyrogenic exotoxin family. The sepe-1 and spe-1 genes differ by only 7 base pairs and the deducted protein sequences differ by 4 amino acids of which two are conservative exchanges (Y225H and E229D) and two are moderate (I112S and N233D). Furthermore, the spe-1 gene is 100% identical to the hypothetical exotoxin gene spe-1 found on prophage PhiHIH 1.1 sequence (NC003157).

Likewise, sepe-m was cloned from isolate FP4223 (serotype M80) and DNA sequence analysis showed strong sequence homology to sepe-m from S. equi and was therefore labelled spe-m. The sepe-m and spe-m genes differ by 13 base pairs and the deducted protein sequences differ by 8 amino acids of which 5 are conservative exchanges (V41I, I51M, H55Y, Y74H and E137D). The 3 non-conservative exchanges are E37V, G175D and N191K.

The calculated molecular weights are 27.4 kD (SePE-L/SPE-L) and 26.2 kD (SePE-M/SPE-M) and the calculated isoelectric points are 6.69 (SePE-L), 6.53 (SPE-L), 6.7 (SePE-M) and 7.1 (SPE-M). The minor differences between SePE-L and SPE-L and between SePE-M and SPE-M suggest that the proteins are orthologues with identical or very similar functions in the two streptococcal species.

A revised streptococcal superantigen family tree, based on primary amino acid sequence homology shows that SePE-L/SPE-L and SePE-M/SPE-M belong to the same subgroup with SMEZ, SPE-C, SPE-G and SPE-J (group A) but build a separate branch within that subgroup (FIG. 5). SePE-L/SPE-L and SePE-M/SPE-M are most closely related to each other (42% sequence identity and 51% sequence simllarity) followed by SPE-C (32%/40%) Table 1.

All novel SAgs contain a zinc-binding motif near the C-terminus that is common among most of the streptococcal SAgs, including all members of group A, and is a prerequisite for binding to the polymorphic MHC class II β-chain via a divalent zinc ion (Roussel A. et al., 1997; Li, Y. et al., 2001). In SPE-C, the zinc-binding residues are H167, H201 and D203 and in SMEZ-2, the zinc ion is bound by residues H162, H202 and D204 (Arcus, V. et al., 2000). The multiple protein sequence alignment (FIG. 1) shows that the most likely candidates for zinc binding are residues H191, H221 and D223 for SePE-L/SPE-L and H186, H217 and D218 for SePE-M/SPE-M.

The most significant difference between the novel SAgs and other streptococcal SAgs is a gap between residues 207 and 208 (SePE-L/SPE-L) and residues 202 and 203 (SePE-M/SPE-M) in the multiple sequence alignment (FIG. 1) In the protein structures of SPE-C, SPE-H and SMEZ-2, the amino acids in this region are assigned to most of the β10-α5 loop and α5 helix (Roussel, A., 1997; Arcus, V. et. al,. 2000). In contrast to loop regions, the structure determining regions, such as β-sheets and α-helices are generally well conserved in SAgs. The missing amino acids in this region suggest a major difference in the SPE-L/SPE-M protein structures compared to other SAgs.

Expression of Recombinant SPEL and rSPE-M

To produce recombinant proteins of SPE-L and SPE-M, the individual subcloned genes (coding for the mature toxins without leader sequence) were transferred to expression vector pET32-3c and transformed into E. coli AD494 (DE3). Fusion proteins of. Trx-SPE-L and Trx-SPE-M were completely soluble and gave yields of about 20 mg per litre. The recombinant toxins were cleaved off from the thioredoxin using protease 3c, while still bound on the column.

The recombinant proteins differ from their native counterparts by four additional N-terminal residues (GPGS) introduced by the BamHI restriction site and the protease 3c recognition site. However, a potential influence on the function of the recombinant proteins seems rather unlikely, as the crystal structures of the closely related proteins SMEZ-2, SPE-C and SPE-H show that the N-terminus is facing away from both the TcR and the MHC class II binding site (Arcus et al, 2000; Roussel et al, 1997).

Eluates containing the purified toxins were collected and analyzed for purity on a 12% SDS-PAGE gel.

T Cell Proliferation and Vβ Specificity

To ensure the native conformation of the purified recombinant toxins, a standard [³H]thymidine incorporation assay was performed to test for their potency to stimulate peripheral blood lymphocytes (PBLs). Both toxins were active on human T cells (FIG. 6). Recombinant SPE-C was included as reference protein.

The half maximal response for rSPE-L and rSPE-M was 1 pg/ml and 10 pg/ml, respectively. No activity was detected at less than 0.1 pg/ml. SPE-L was as potent as rSPE-C that also had a P50 value of 1 pg/ml. Recombinant SPE-M was less active than rSPE-C, but still more potent than the previously described SPE-H (50 pg/ml) (Proft, T. et al., 1999).

Vβ Specificity of Recombinant Toxins.

The human TcR Vβ specificity of the recombinant toxins was determined by multiprimer anchored PCR and dot blot analysis using a panel of 21 human Vβ DNA regions. The Vβ enrichment after stimulation with toxin was compared to the Vβ profile of PBLs stimulated with the unspecific T cell mitogen ConA (Table 2).

Table 2. Vβ Specificity of Recombinant Toxins on Human PBLs.

Human PBLs were incubated with 20 pg/ml of recombinant toxin for 4 d. Relative enrichment of Vβ cDNAs was analyzed from RNA of toxin stimulated and ConA stimulated PBLs by anchored primer PCR and reverse dot-blot to a panel of 22 different Vβ cDNAs. The figures represent the percentage of each Vβ with respect to total Cβ. Significant responses are underlined.

TABLE 2

| | Percentage of Vβ enrichment | | |
|---|---|---|---|
| Vβ | ConA | SPE-L | SPE-M |
| 1.1 | 0.1 | 8.44 | 6.75 |
| 2.1 | 3.94 | 4.46 | 4.2 |
| 3.1 | 19.57 | 6.53 | 9.83 |
| 4.1 | 0.12 | 0.98 | 1.41 |
| 5.1 | 1.82 | 2.78 | 3.69 |
| 5.3 | 12.06 | 15.2 | 13.96 |
| 5.8 | 3.31 | 4.49 | 4.43 |
| 6.3 | 1.03 | 1.57 | 2.15 |
| 6.4 | 0.63 | 1.41 | 1.47 |
| 6.9 | 5.96 | 4.85 | 4.92 |
| 7.3 | 0.84 | 0.94 | 1.1 |
| 7.4 | 2.31 | 1.71 | 1.47 |
| 8.1 | 0.3 | 0.68 | 0.79 |
| 9.1 | 1.34 | 1.78 | 2.05 |
| 12.3 | 15.36 | 7.76 | 8.93 |
| 12.5 | 1.17 | 1.38 | 1.94 |
| 14.1 | 0.3 | 0.79 | 0.95 |
| 15.1 | 2.26 | 3.92 | 4.25 |
| 17.1 | −0.07 | 1.05 | 1.12 |
| 18.1 | 1.51 | 2.54 | 3.13 |
| 22a | 1.1 | 2.09 | 2.27 |
| 23.1 | 0.45 | 1.39 | 1.41 |
| total % | 71.6 | 78 | 82.2 |

Both toxins showed very similar results with Vβ1.1 TcR being the primary target. Initial results indicated T cells carrying the Vβ1.1 TcR were stimulated >50 times and >60 times more with rSPE-L or rSPE-M, respectively, than with ConA. Further results showed that T cells carrying the Vβ1.1 TcR were stimulated >80 times and nearly 70 times more with rSPE-L or rSPE-M, respectively, than with ConA (Table 2).

Interestingly, the total of the Vβs stimulated with rSPE-L or rSPE-M was only 78% and 82%, respectively, suggesting that one or more additional Vβ were stimulated which are not represented in the panel. To further investigate this possibility, the Vβ cDNAs from rSPE-L and rSPE-M stimulated T cells were cloned into a pBluescript vector and the DNA sequences of 10 randomly selected clones were analyzed. Five out of 5 cDNAs from rSPE-L stimulated T cells and 4/5 cDNAs from rSPE-M stimulated T cells were identified as Vβ1.1. One rSPE-M expanded cDNA was identified as Vβ5.8. However, Vβ5.8 was not significantly enriched by either toxin (around 4.5%) compared to ConA stimulated cells (3.3%) Table 2.

MHC Class I Binding

As already mentioned, the protein sequences of-SPE-L and SPE-M contain the highly conserved zinc binding motif that is required for binding to the polymorphic MHC class II β-chain.

In an attempt to determine the orientation of the toxins on MHC class II competition binding experiments were performed. The recombinant toxins were radiolabelled and tested with excess of unlabelled toxin for binding to LG-2 cells. Recombinant SEB, RTSST and rSPE-C were used as reference proteins. SPE-C binds exclusively to the MHC class II β-chain using the zinc binding motif (Li et al, 1997; Li et al, 2001), while SEB and TSST both exclusively bind to the MHC class II α-chain in a different, zinc-independent binding mode (Acharya et al, 1994; Hurley et al, 1995; Seth et al, 1994). The results are shown in FIG. 7. Binding of radiolabelled rSPE-L or rSPE-M was inhibited with excess amounts of unlabelled rSPE-C, rSPE-L and rSPE-M. In contrast, excess amounts of unlabelled rSEB or rTSST did not influence binding of radiolabelled rSPE-L and rSPE-M. These results suggest that both, SPE-L and SPE-M, bind MHC class II exclusively at the β-chain in a zinc-dependent binding mode which appears to be conserved among the SPE-C subfamily of the SAg family (ie group A members of the SAg family).

Sero-Conversion Against SPE-L and SPE-M

SAgs are generally produced and secreted in minimal (often nanomolar) amounts by the bacteria. Detection of the toxins by immunological methods, like Western blot or ELISA, is often not possible or depends on high affinity monoclonal antibodies. An alternative method is the detection of toxin specific antibodies in human sera. Specific antibodies against pathogen factors are often produced after bacterial infections, even when non-severe, and can protect the individual from further encounter with bacteria producing these toxins.

20 sera from healthy donors were analysed for antibodies against SPE-L and SPE-M. ¹²⁵I-labelled toxin was used to bind specific IgG antibodies, which were then precipitated with staphylococcal cell bound protein A (IgG receptor protein) (FIG. 8). 7 subjects were identified with sero-conversion against SPE-L, of which 4 had a strong titer, 1 had a moderate titer and 2 had a low titer. 6 serum samples had significant antibody titers against SPE-M (2 high, 3 moderate and 1 low). 4 sera showed conversion against both toxins. BSA and rabbit antibodies against SPE-C and SMEZ showed no binding to labelled SPE-L or SPE-M indicating the strong specificity of the antibodies.

These results indicate that both, SPE-L and SPE-M, are actually expressed and secreted by some streptococcal isolates and that these toxins represent potential pathogen factors.

### INDUSTRIAL APPLICATION

The superantigens of the invention, polynucleotides which encode them and antibodies which bind them have numerous applications. A number of these are discussed above (including Streptococci subtyping, diagnostic applications and therapeutic applications) but it will be appreciated that these are but examples. Other applications will present themselves to those skilled in the art and are in no way excluded from the scope of the invention.

It will also be appreciated that the foregoing examples are illustrations of the invention. The invention may be carried out with the numerous variations and modifications as will be apparent to those skilled in the art. For example, a native superantigen may be replaced by a synthetic superantigen with on or more deletions, insertions and/or substitutions relative to the corresponding natural superantigen, provided that the superantigen activity is retained. Likewise there are many variations in the way in which the invention can be used in other aspects of it.

Throughout this specification, unless the context requires otherwise, the words “comprise”, “comprising” and the like, are to be construed in an inclusive sense as opposed to an exclusive sense, that is to say, in the sense of “including, but not limited to”.

The reference to any prior art in this specification is not, and should not be taken as, an acknowledgment or any form of suggestion that the prior art forms part of the common general knowledge in New Zealand or in any other country.

### REFERENCES

Marrack, P., and J. Kappler. 1990. The staphylococcal enterotoxins and their relatives. Science 248:705-711.

Huber, B. T., P. N. Hsu, and N. Sutkowski. 1996. Virus-encoded superantigens. Microbiol. Rev. 60, no. 3:473-82.

Alouf, J. E., H. Knoell, and W. Koehler. 1991. The family of mitogenic, shock-inducing and superantigenic toxins from staphylococci and streptococci. Sourcebook of bacterial protein toxins., eds. J. E. Alouf and J. H. Freer. Academic Press, San Diego. 367-414 pp.

Betley, M. J., D. W. Borst, and L. B. Regassa. 1992. Staphylococcal enterotoxins, toxic shock syndrome toxin and streptococcal exotoxins: a comparative study of their molecular biology. Chem. Immunol. 55:1-35.

Ren, K., J. D. Bannan, V. Pancholi, A. L. Cheung, J. C. Robbins, V. A. Fischetti, and J. B. Zabriskie. 1994. Characterization and biological properties of a new staphylococcal exotoxin. J. Exp. Med. 180, no. 5:1675-83.

Munson, S. H., M. T. Tremaine, M. J. Betley, and R. A. Welch. 1998. Identification and Characterization Of Staphylococcal Enterotoxin Types G and I From Staphylococcus Aureus. Infect. Immun. 66, no. 7:3337-3348.

Herman, A., J. W. Kappler, P. Marrack, and A. M. Pullen. 1991. Superantigens: mechanism of T-cell stimulation and role in immune responses. Annu. Rev. Immunol. 9:745-772.

Janeway, C. J., J. Yagi, P. J. Conrad, M. E. Katz, B. Jones, S. Vroegop, and S. Buxser. 1989. T-cell responses to Mls and to bacterial proteins that mimic its behavior. Immunol. Rev. 107:61-68.

Fast, D. J., P. M. Schlievert, and R. D. Nelson. 1989. Toxic shock syndrome-associated staphylococcal and streptococcal pyrogenic toxins are potent inducers of tumor necrosis factor production. Infect. Immun. 57, no. 1:291-4.

Gerlach et al. 2000. FEMS Microbiol Letters. 188;pp 153-163.

Kotzin, B. L., D. Y. Leung, J. Kappler, and P. Marrack. 1993. Superantigens and their potential role in human disease. Adv. Immunol. 54, no. 99:99-166.

Bohach, G. A., D. J. Fast, R. D. Nelson, and P. M. Schlievert. 1990. Staphylococcal and streptococcal pyrogenic toxins involved in toxic shock syndrome and related illnesses. Crit. Rev. Microbiol. 17, no. 4:251-72.

Weeks, C. R., and J. J. Ferretti. 1986. Nucleotide Sequence of the Type A Streptococcal Exotoxin (Erythrogenic Toxin) Gene from Streptococcus pyogenes Bacteriophage T12. Infect. Immun. 52:144-150.

Goshorn, S. C., G. A. Bohach, and P. M. Schlievert. 1988. Cloning and characterization of the gene, speC, for pyrogenic exotoxin type C from Streptococcus pyogenes. Mol. Gen. Genet. 212, no. 1:66-70.

Mollick, J. A., G. G. Miller, J. M. Musser, R. G. Cook, D. Grossman, and R. R. Rich. 1993. A novel superantigen isolated from pathogenic strains of Streptococcus pyogenes with aminoterminal homology to staphylococcal enterotoxins B and C. J. Clin. Invest. 92, no. 2:710-9.

Van Den Busche, R. A., J. D. Lyon, and G. A. Bohach. 1993. Molecular evolution of the staphylococcal and streptococcal pyrogenic toxin gene family. Mol. Phylogenet. Evol. 2:281-292.

Dellabona, P., J. Peccoud, J. Kappler, P. Marrack, C. Benoist, and D. Mathis. 1990. Superantigens interact with MHC class II molecules outside of the antigen groove. Cell 62, no. 6:1115-21.

Fraser, J. D. 1989. High-affinity binding of staphylococcal enterotoxins A and B to HLA-DR. Nature 339, no. 6221:221-3.

Fleischer, B., and H. Schrezenmeier. 1988. T cell stimulation by staphylococcal enterotoxins. Clonally variable response and requirement for major histocompatibility complex class II molecules on accessory or target cells. J. Exp. Med. 167, no. 5:1697-707.

Mollick, J. A., R. G. Cook, and R. R. Rich. 1989. Class II MHC molecules are specific receptors for staphylococcus enterotoxin A. Science 244, no. 4906:817-20.

Schad, E. M., I. Zaitseva, V. N. Zaitsev, M. Dohlsten, T. Kalland, P. M. Schlievert, D. H. Ohlendorf, and L. A. Svensson. 1995. Crystal structure of the superantigen staphylococcal enterotoxin type A. EMBO J. 14, no. 14:3292-301.

Swaminathan, S., W. Furey, J. Pletcher, and M. Sax. 1992. Crystal structure of staphylococcal enterotoxin B, a superantigen. Nature 359, no. 6398:801-6.

Papageorgiou, A. C., K. R. Acharya, R. Shapiro, E. F. Passalacqua, R. D. Brehm, and H. S. Tranter. 1995. Crystal structure of the superantigen enterotoxin C2 from Staphylococcus aureus reveals a zinc-binding site. Structure 3, no.8:769-79.

Sundstrom, M., L. Abrahmsen, P. Antonsson, K. Mehindate, W. Mourad, and M. Dohlsten. 1996. The crystal structure of staphylococcal enterotoxin type D reveals Zn2+-mediated homodimerization. EMBO J. 15, no. 24:6832-40.

Acharya, K. R., E. F. Passalacqua, E. Y. Jones, K. Harlos, D. I. Stuart, R. D. Brehm, and H. S. Tranter. 1994. Structural basis of superantigen action inferred from crystal structure of toxic-shock syndrome toxin-1. Nature 367, no. 6458:94-7.

Roussel, A., B. F. Anderson, H. M. Baker, J. D. Fraser, and E. N. Baker. 1997. Crystal structure of the streptococcal superantigen SPE-C: dimerization and zinc binding suggest a novel mode of interaction with MHC class II molecules. Nat. Struct. Biol. 4, no. 8:635-43.

Kim, J., R. G. Urban, J. L. Strominger, and D. C. Wiley. 1994. Toxic shock syndrome toxin-1 complexed with a class II major histocompatibility molecule HLA-DR1. Science 266, no. 5192:1870-4.

Hurley, J. M., R. Shimonkevitz, A. Hanagan, K. Enney, E. Boen, S. Malmstrom, B. L. Kotzin, and M. Matsumura. 1995. Identification of class II major histocompatibility complex and T cell receptor binding sites in the superantigen toxic shock syndrome toxin 1. J. Exp. Med. 181, no. 6:2229-35.

Seth, A., L. J. Stern, T. H. Ottenhoff, I. Engel, M. J. Owen, J. R. Lamb, R. D. Klausner, and D. C. Wiley. 1994. Binary and ternary complexes between T-cell receptor, class II MHC and superantigen in vitro. Source (Bibliographic Citation): Nature 369, no. 6478:324-7.

Li, P. L., R. E. Tiedemann, S. L. Moffat, and J. D. Fraser. 1997. The superantigen streptococcal pyrogenic exotoxin C (SPE-C) exhibits a novel mode of action. J. Exp. Med 186, no. 3:375-83.

Hudson, K. R., R. E. Tiedemann, R. G. Urban, S. C. Lowe, J. L. Strominger, and J. D. Fraser. 1995. Staphylococcal enterotoxin A has two cooperative binding sites on major histocompatibility complex class II. J. Exp. Med. 182, no. 3:711-20.

Kozono, H., D. Parker, J. White, P. Marrack, and J. Kappler. 1995. Multiple binding sites for bacterial superantigens on soluble class II MHC molecules. Immunity 3, no. 2:187-96.

Tiedemann, R. E., and J. D. Fraser. 1996. Cross-linking of MHC class II molecules by staphylococcal enterotoxin A is essential for antigen-presenting cell and T cell activation. J. Immunol. 157, no. 9:3958-66.

Braun, M. A., D. Gerlach, U. F. Hartwig, J. H. Ozegowski, F. Romagne, S. Carrel, W. Kohler, and B. Fleischer. 1993. Stimulation of human T cells by streptococcal “superantigen” erythrogenic toxins (scarlet fever toxins). J. Immunol. 150, no. 6:2457-66.

Kline, J. B., and C. M. Collins. 1997. Analysis of the interaction between the bacterial superantigen streptococcal pyrogenic exotoxin A (SpeA) and the human T-cell receptor. Mol. Microbiol. 24, no. 1:191-202.

Fleischer, B., A. Necker, C. Leget, B. Malissen, and F. Romagne. 1996. Reactivity of mouse T-cell hybridomas expressing human Vbeta gene segments with staphylococcal and streptococcal superantigens. Infect. Immun. 64, no. 3:987-94.

Toyosaki, T., T. Yoshioka, Y. Tsuruta, T. Yutsudo, M. Iwasaki, and R. Suzuki. 1996. Definition of the mitogenic factor (MF) as a novel streptococcal superantigen that is different from streptococcal pyrogenic exotoxins A, B, and C. Eur. J. Immunol. 26, no. 11:2693-701.

Kamezawa, Y., T. Nakahara, S. Nakano, Y. Abe, J. Nozaki-Renard, and T. Isono. 1997. Streptococcal mitogenic exotoxin Z, a novel acidic superantigenic toxin produced by a T1 strain of Streptococcus pyogenes. Infect. Immun. 65, no. 9:3828-33.

Hudson, K. R., H. Robinson, and J. D. Fraser. 1993. Two adjacent residues in Staphylococcal enterotoxins A an E determine Tcell receptor V beta specificity. J. Exp. Med. 177:175-185.

Kraulis, P. J. 1991. MOLSCRIPT”: a program to produce both detailed and schematic plots of protein structures. J. Appl. Critallography 24:946-950.

Cunningham, B. C., P. Jhurani, P. Ng, and J. A. Wells. 1989. Receptor and Antibody epitopes in human growth hormone identified by homologue scanning mutagenesis. Science 243:1330-1336.

Fields, B. A., E. L. Malchiodi, H. Li, X. Ysern, C. V. Stauffacher, P. M. Schlievert, K. Karjalainen, and R. A. Mariuzza. 1996. Crystal structure of a T-cell receptor beta-chain complexed with a superantigen [see comments]. Nature 384, no. 6605:188-92.

Wen, R., G. A. Cole, S. Surman, M. A. Blackman, and D. L. Woodland. 1996. Major histocompatibility complex class II-associated peptides control the presentation of bacterial superantigens to T cells. J. Exp. Med 183, no. 3:1083-92.

Thibodeau, J., I. Cloutier, P. M. Lavoie, N. Labrecque, W. Mourad, T. Jardetzky, and R. P. Sekaly. 1994. Subsets of HLA-DR1 molecules defined by SEB and TSST-1 binding. Science 266, no. 5192:1874-8.

Abe, J., B. L. Kotzin, K. Jujo, M. E. Melish, M. P. Glode, T. Kohsaka, and D. Y. Leung. 1992. Selective expansion of T cells expressing T-cell receptor variable regions V beta 2 and V beta 8 in Kawasaki disease. PNAS 89, no. 9:4066-70.

Kawasaki, T. 1967. Acute febrile mucocutaneous syndrome with lymphoid involvement with specific desquamation of the fingers and toes in children. Jpn. J. Allergol. 16:178.

Leung, D. Y., R. C. Giorno, L. V. Kazemi, P. A. Flynn, and J. B. Busse. 1995. Evidence for superantigen involvement in cardiovascular injury due to Kawasaki syndrome. J. Immunol. 155, no. 10:5018-21.

Cockerill, F. R., R. L. Thompson, J. M. Musser, P. M. Schlievert, J. Talbot, K. E. Holley, W. S. Harmsen, D. M. Ilstrup, P. C. Kohner, M. H. Kim, B. Frankfort, J. M. Manahan, J. M. Steckelberg, F. Roberson, and W. R. Wilson. 1998. Molecular, Serological, and Clinical Features Of 16 Consecutive Cases Of Invasive Streptococcal Disease. Clin. Infect. Dis. 26, no. 6:1448-1458.

Kapur, V., K. B. Reda, L. L. Li, L. J. Ho, R. R. Rich, and J. M. Musser. 1994. Characterization and distribution of insertion sequence IS1239 in Streptococcus pyogenes. Gene 150, no. 1:135-40.

T. Proft, S. L. Moffatt, C. J. Berkahn, and J. D. Fraser (1999). Identification and characterisation of novel superantigens from Streptoccocus pyogenes. Journal of Experimental Medicine 189, No. 1:89-102.

T. Maniatis, E. F. Fritsch, and J. Sambrook. (1989). Molecular cloning: a laboratory manual. Cold Spring Harbour Laboratory, Cold Spring Harbour, N.Y., USA.

B. A. Roe, S. P. Linn, L. Song, X. Yuan, S. Clifton, M. McShan and J. Ferretti, (1999). Str. Pyogenes M1 genome sequencing project at Oklahoma University. Web: http://www.genome.ou.edu.

Thompson, J. D., Higgins, D. G. and Gibson, T. J., 1994. CLUSTAL W: improving the sensitivity of progressive multiple sequence alignment. Nucleic Acids Research 22:4673-4680.

Martin, D. R., Voss, L. M., Walker, S. J. and Lennon, D. 1994. Acute rheumatic fever in Auckland, New Zealand: spectrum of associated Group A streptococci different from expected. Pediatr. Infect. Dis. J. 13, no. 4:264-269.

Walker, P. A., Leong, L. E., Ng, P. W., Tan, S. H., Waller, S., Murphy, D. and Porter, A. G. 1994. Efficient and rapid affinity purification of proteins using recombinant fusion proteases. Bio/Technology 12, no. 6:601-605.

Page, R. D. M. 1996. TREEVIEW: An application to display phylogenetic trees on personal computers. Computer Applications in the Biosciences 12:357-358.

Arcus, V. L., Proft, T., Sigrell, J. A., Baker, H. M., Fraser, J. D. and Baker, E. N. 2000. Conservation and variation in superantigen structure and activity highlighted by the three-dimensional structures of two new superantigens from Streptococcus pyogenes. Journal of Molecular Biology 299, no. 1:157-168.

Artiushin, S. C., Timoney, J. F., Sheoran, A. S. and Muthupalani, S. K. 2002. Characterization and immunogenecity of pyrogenic mitogens SePE-H and SePE-I of Streptococcus equi. Microbial Path 32:71-85.

Li, Y., Li, H., Dimasi, N., McCormick, J. K., Martin, R., Schnuck, P., Schlievert, P. M. and Mariuzza, R. A. 2001. Crystal Structure of a Superantigen Bound to the High-Affinity, Zinc-Dependent Site on MHC Class II. Immunity 14:93-104.

Proft, T., S. L. Moffatt, K. D. Weller, A. Paterson, D. Martin, and J. D. Fraser. 2000. The streptococcal superantigen SMEZ exhibits wide allelic variation, mosaic structure, and significant antigenic variation. J. Exp. Med. 191:1765-1776.

Thompson, J. D., D. G. Higgins, and T. J. Gibson. 1994. CLUSTAL W: improving the sensitivity of progressive multiple sequence alignment. Nucl. Acids Res. 22:4673-4680.

Nielsen, H., J. Engelbrecht, S. Brunak, and G. von Heijne. 1997. Identification of prokaryotic and eukaryotic signal peptides and prediction of their cleavage sites. Prot. Eng. 10: 1-6

1. A superantigen selected from either SPE-L or SPE-M, or a functionally equivalent variant thereof.

2. A superantigen as claimed in claim 1 comprising the amino acid sequence of SEQ ID NO: 2.

3. A superantigen as claimed in claim 1 comprising the amino acid sequence of SEQ ID NO:4.

4. A superantigen as claimed in claim 1 encoded by the nucleic acid of SEQ ID NO:1.

5. A superantigen as claimed in claim 1 encoded by the nucleic acid of SEQ ID NO:3.

6. A nucleic acid encoding a superantigen selected from either SPE-L or SPE-M, or a functionally equivalent variant thereof.

7. A nucleic acid as claimed in claim 6 comprising at least the nucleic acid sequence of SEQ ID NO:1.

8. A nucleic acid as claimed in claim 6 comprising at least the nucleic acid sequence of SEQ ID NO:3.

9. A nucleic acid comprising the sequence SEQ ID NO:1, or a variant thereof.

10. A nucleic acid comprising the sequence SEQ ID NO:3, or a variant thereof.

11. A nucleic acid selected from the group consisting of:
SEQ ID NO: 5
SEQ ID NO: 6
SEQ ID NO: 7
SEQ ID NO: 8
SEQ ID NO: 9
SEQ ID NO: 10
SEQ ID NO: 11
SEQ ID NO: 12

12. Nucleic acid constructs comprising one or more of the nucleic acids of claims 6 to 11.

13. A method of determining in a sample the presence or absence of the superantigens SPE-L and/or SPE-M, or functional equivalents thereof, comprising at least the steps of:
Providing a sample to be tested; and
Determining whether or not either or both of the superantigens, or their functional equivalents are present.

14. A method of determining in a sample the presence or absence of nucleic acid molecules encoding of the superantigens SPE-L and/or SPE-M, or functional equivalents thereof, comprising at least the steps of:
Providing a sample to be tested; and
Determining whether or not nucleic acid molecules encoding either or both of the superantigens, or their functional equivalents are present.

15. A method of subtyping Streptococcus in a sample, the method comprising at least the steps of:
Providing a sample to be tested; and
Determining whether or not either or both of SPE-L or SPE-M, or their functional equivalents are present.

16. A method of subtyping Streptococcus in a sample, the method comprising at least the steps of:
Providing a sample to be tested; and
Determining whether or not nucleic acid molecules encoding either or both of the superantigens, or their functional equivalents are present.

17. A method as claimed in claim 15 or 16 wherein the method is for the purpose of subtyping M-types of S.pyogenes.

18. A method of diagnosing infection of a subject with S.pyogenes, the method comprising at least the steps of:
Providing a sample from a subject to be tested; and,
Determining whether or not either or both of SPE-L or SPE-M, or their functional equivalents are present.

19. A method of diagnosing infection of a subject with S.pyogenes, the method comprising at least the steps of:
Providing a sample from a subject to be tested; and,
Determining whether or not nucleic acid molecules encoding either or both of the superantigens, or their functional equivalents are present.

20. A method as claimed in claim 18 or 19 wherein the method is for the purpose of diagnosing infection of a subject with S.pyogenes M-types M28, M41, M56, M59 and/or M89.

21. A method as claimed in claim 18 or 19 wherein the method is for the purpose of diagnosing infection of a subject with Spyogenes M-types M80 and/or M92.

22. A method as claimed in any one of claims 18 to 21 wherein the sample is chosen from one or more of:
Saliva;
Blood; and
Tissue.

23. A method as claimed in claims 14, 16 or 19 wherein the method employs PCR.

24. A method as claimed in claim 23 wherein the method employs forward primers chosen from SEQ ID NO: 5 and SEQ ID NO:9, and reverse primers chosen from SEQ ID NO: 6 and SEQ ID NO:10.

25. A method as claimed in claim 23 wherein the method employs forward primers chosen from SEQ ID NO: 7 and SEQ ID NO:11, and reverse primers chosen from SEQ ID NO: 8 and SEQ ID NO:12.

26. A method as claimed in claims 14, 16 or 19 wherein the method employs nucleic acid hybridisation with one or more nucleic acid probes.

27. A method as claimed in claim 26 wherein nucleic acid hybridisation occurs by Southern blotting.

28. A method as claimed in claim 26 or 27 wherein the one or more nucleic acid probes is chosen from the group consisting SEQ ID NOs:1, 3, 5 to 12, or variants thereof.

29. A method of determining whether or not a subject has been exposed to SPE-L and/or SPE-M comprising at least the steps of:
Providing a sample from a subject to be tested;
Determining whether or not the sample contains antibodies specific to SPE-L and/or SPE-M.

30. A method as claimed in claim 29 wherein the sample comprises peripheral blood lymphocytes.

31. A method as claimed in claim 29 or 30 wherein the antibodies are neutralising antibodies.

32. A method as claimed in any one of claims 29 to 31 wherein a T cell stimulation assay using SPE-L and/or SPE-M or their functional equivalents is used to determine whether or not the sample contains antibodies specific to SPE-L and/or SPE-M.

33. A method as claimed in any one of claims 29 to 32 wherein the method is performed to determine whether or not a subject has been exposed to Spyogenes M-types M28, M41, M56, M59, M89, M80 and/or M92.

34. A construct which comprises a superantigen or variant thereof as claimed in claim 1 and a cell-targeting molecule.

35. A construct as claimed in claim 34 in which said cell-targeting molecule specifically binds a tumour cell.

36. A construct as claimed in claim 34 or 35 in which said cell-targeting molecule is an antibody.

37. A pharmaceutical composition which comprises at least a construct as claimed in any one of claims 34 to 36, optionally in association with one or more pharmaceutically acceptable carriers, diluents or excipients.

38. An antibody which binds SPE-L or a functionally equivalent variant thereof.

39. An antibody which binds SPE-M or a functionally equivalent variant thereof.

40. A nucleic acid molecule which hybridises to a polynucleotide of claim 9.

41. A nucleic acid molecule which hybridises to a polynucleotide of claim 10.

42. A kit which includes a nucleic acid molecule as claimed in any one of claims 6 to 11.


SEQUENCE LISTING (12 sequences)

SEQ ID NO: 1 - DNA, 702 bp, streptococcus pyogenes
gatacgtacaatacaaatgatgttagaaatccaaggaacatatatgctcctagatatgataaagacgaaattttggataatagaagattaaaagaaatatataataaagaaattattgaaaaaaataatatatcgataaatgccaaacaaggaacgcaattgatttttaatacggatgaaaatactacagtttggaatgataacacttttaagaaagtcatatctagtaatctttctccttcacaggaaagaatgtttaatgttggtgatcatgtgaatatttttgctatagtaaagtcatatcatgttgtatgcaaggaacaattcaattatagtgatgggggaataataaaaacaagtgatgtaaaaccagaagaaaaagcaatttatattaatatttttggtgaaaaagaattacgaacattaacagctaaagataagattacctttaaaaataatattgtaactcttcaggagattgatgttagacttaggaaaagtttgatgggggacagcaaaataaaattgtatgagtacgattctttgtataaaaaagggttttgggatattcattataaagacggtggcattagacacaccaatttatttacttaccccgactatacagataatgaaacgattgatatgagtaaagttagtcactttgatgttcacttaaacgaagatttttctaaagattag

SEQ ID NO: 2 - protein, 233 aa, Streptococcus pyogenes
DTYNTNDVRNPRNIYAPRYDKDEILDNRRLKEIYNKEIIEKNNISINAKQGTQLIFNTDENTTVWNDNTFKKVISSNLSPSQERMFNVGDHVNIFAIVKSYHVVCKEQFNYSDGGIIKTSDVKPEEKAIYINIFGEKELRTLTAKDKITFKNNIVTLQEIDVRLRKSLMGDSKIKLYEYDSLYKKGFWDIHYKDGGIRHTNLFTYPDYTDNETIDMSKVSHFDVHLNEDFSKD

SEQ ID NO: 3 - DNA, 684 bp, Streptococcus pyogenes
gaggggactattaatattaaggatatatactctccaaggtgggatgtagataaaacattatcccctactaccttaagagaaatttataatagagatactataaaaaaagtgaataaacccattactggaaaaagagggacgcaagttattatggatgctcagtataaaactaaagtatgggaatttgatgattataattttataatatcaagtaacctacacccatctgtagaaggtaaatttaatgttggagataatgtcgatgtttttggtcttgcattatcagctgaagtattttcaaaagatcaaatacattcaatcaatggtggtctcgttaaagttaatgagagaaaaggagccggaaaaacgatttacatgaacgtttttattgatgggcataaaaaagatgatacctcgaaatataaaataacttttgaaaaatctcccgttaccttccaggaagttgatgttagattgagaaaatcatttatgctgaacgatgaaataaaactttatcagtatgattccaaagttctatctggaaactgggaatttcacggctcaggcgagaaagaggaaggtgctgacttatttaaatacccagattatagatataataatttgatagatatagacaaaaagagtcatattgatgtctatttattcacaaacaaagaaaattag

SEQ ID NO: 4 - protein, 227 aa, Streptococcus pyogenes
EGTINIKDIYSPRWDVDKTLSPTTLREIYNRDTIKKVNKPITGKRGTQVIMDAQYKTKVWEFDDYNFIISSNLHPSVEGKFNVGDNVDVFGLALSAEVFSKDQIHSINGGLVKVNERKGAGKTIYMNVFIDGHKKDDTSKYKITFEKSPVTFQEVDVRLRKSFMLNDEIKLYQYDSKVLSGNWEFHGSGEKEEGADLFKYPDYRYNNLIDIDKKSHIDVYLFTNKEN

SEQ ID NO: 5 - DNA, 27 bp, Streptococcus pyogenes
gcggatccgatacgtacaatacaaatg

SEQ ID NO: 6 - DNA, 22 bp, Streptococcus pyogenes
gcgaattcaatagcattcgacc

SEQ ID NO: 7 - DNA, 29 bp, Streptococcuc pyogenes
gcggatccgaggggactattaatattaag

SEQ ID NO: 8 - DNA, 25 bp, Streptococcus pyogenes
gcgaattcggtttcttgatactaac

SEQ ID NO: 9 - DNA, 19 bp, Streptococcus pyogenes
gatacgtacaatacaaatg

SEQ ID NO: 10 - DNA, 14 bp, Streptococcus pyogenes
aatagcattcgacc

SEQ ID NO: 11 - DNA, 21 bp, Streptococcus pyogenes
gaggggactattaatattaag

SEQ ID NO: 12 - DNA, 17 bp, Streptococcus pyogenes
ggtttcttgatactaac